(19) United States
(12) Patent Application Publication
(10) Pub. No.: US 2024/0325312 A1
(43) Pub. Date: Oct. 3, 2024

(54) Composition comprising cannabidiol and hydroxychloroquine in a fixed dose combination capsule

(71) Applicant: Incannex Healthcare Limited, Norwest, New South Wales (AU)

(72) Inventors: Mark Robert Bleackley, South Morang, Victoria (AU); Joel Bradley Latham, North Kellyville, New South Wales (AU)

(21) Appl. No.: 18/576,218

(22) PCT Filed: Jul. 13, 2022
(86) PCT No.: PCT/AU2022/050731

§ 371 Date: Jan. 3, 2024

(30) Foreign Application Priority Data
Jul. 15, 2021 (AU) 2021902170

(51) Int. Cl. A61K 9/48; A61K 31/00; A61K 31/4706; A61P 29/00

(52) U.S. Cl. CPC A61K 9/4808; A61K 9/4825; A61K 31/4706; A61K 31/658; A61P 29/00

(57) ABSTRACT

The present disclosure relates generally to compositions comprising a synergistic combination of cannabidiol (CBD) or a pharmaceutically acceptable salt or derivative thereof and hydroxychloroquine or a pharmaceutically acceptable salt thereof, which beneficially enables simultaneous administration of the two active pharmaceutical ingredients (APIs) in a single dosage form. In an embodiment, the present disclosure also relates to methods and uses of the composition for the treatment of an inflammatory condition.

### RELATED APPLICATIONS

This application claims priority from Australian Provisional Patent Application No. 2021902170 filed on 15 Jul. 2021, the entire content of which is hereby incorporated by reference.

### FIELD

The present disclosure relates generally to compositions comprising a synergistic combination of cannabidiol (CBD) or a pharmaceutically acceptable salt or derivative thereof and hydroxychloroquine or a pharmaceutically acceptable salt thereof, which beneficially enables simultaneous administration of the two active pharmaceutical ingredients (APIs) in a single dosage form. In an embodiment, the present disclosure also relates to methods and uses of the composition for the treatment of an inflammatory condition.

### BACKGROUND

Fixed-dose combination (FDC) pharmaceutical products comprising multiple active pharmaceutical ingredients (APIs) are often difficult to formulate where active ingredients are incompatible, that is, the APIs deleteriously react with one another when combined in desired levels of concentrations. FDCs also require significant product formulation and manufacturing optimisation to address stability, dose differential and physical and chemical compatibility.

In the context of FDCs comprising cannabidiol (CBD), these limitations can become more onerous due to issues with bioavailability, variable pharmacokinetic profiles, possible polymorphisms and a higher risk of drug-drug interactions (DDIs) associated with CBD. However, recent studies have shown that CBD can synergize with other APIs for the treatment of a range of different conditions, including traumatic brain injury (e.g., WO 2021/062481), glioblastoma multiform (GBM; Lopez-Valero, 2018, Biochemical Pharmacology, 157: 266-274) and Gram-positive bacterial infections (Wassmann, 2020, Scientific Reports, 10, Article No. 4112).

In most cases, synergism between CBD and one or more additional APIs occurs where the two or more APIs are delivered to a subject simultaneously. Using standard methods in the art, it would be expected that the formulation and development of any such single dosage forms or FDCs would require extensive consideration of the physical characteristics of the APIs, the mode of delivery, the flow properties of the composition, the excipient compatibility, the uniformity in production and the release profile to ensure that the pharmacokinetic properties of the APIs are maintained (or enhanced) and/or impart stability to the composition such that it can have an acceptable shelf life.

Therefore, there remains an urgent need for the development of pharmaceutical compositions that enable the simultaneous delivery of CBD and one or more additional APIs in a single dosage form (e.g., a FDC) regardless of physical-chemical compatibility and/or stability liabilities.

### SUMMARY

In an aspect of the present disclosure, there is provided a composition comprising:
a. cannabidiol (CBD) or a pharmaceutically acceptable salt or derivative thereof; and
b. a solid dosage form of hydroxychloroquine or a pharmaceutically acceptable salt thereof,
   wherein the CBD or a pharmaceutically acceptable salt or derivative thereof is encapsulated by a first capsule and the solid dosage form of hydroxychloroquine or a pharmaceutically acceptable salt thereof is encapsulated by a second capsule, wherein the second capsule is at least partially encapsulated by the first capsule.

### BRIEF DESCRIPTION OF THE FIGURES

Various examples and embodiments of the invention are described herein, by way of non-limiting example only, with reference to the following figures.

FIG. 1 is a schematic representation of the composition comprising a first capsule and a second capsule, in which the second capsule comprises a tablet solid dosage form of hydroxychloroquine and is encapsulated by the first capsule.

FIG. 2 is a schematic representation of the composition comprising a first capsule and a second capsule, in which the second capsule comprises a particulate solid dosage form of hydroxychloroquine (e.g., a powder, a microgranule, a nanoparticle).

FIG. 3 shows that CBD and hydroxychloroquine are effective for the treatment of mice with pulmonary inflammation. (A) A photographic representation of normal lung tissue in sham-treated control mice; (B) A photographic representation of lung tissue from vehicle control mice; (C) A photographic representation of lung tissue from mice treated with CBD (1 mg/kg); (D) A photographic representation of lung tissue from mice treated with hydroxychloroquine (2.5 mg/kg); (E) A photographic representation of lung tissue from mice treated with CBD (1 mg/kg) and hydroxychloroquine (2.5 mg/kg); (F) A photographic representation of lung tissue from mice treated with CBD (10 mg/kg); (G) A photographic representation of lung tissue from mice treated with hydroxychloroquine (25 mg/kg); and (H) A photographic representation of lung tissue from mice treated with CBD (10 mg/kg) and hydroxychloroquine (25 mg/kg). All images shown at 100× magnification, and stained with hematoxylin and eosin (H&E). Arrows indicate inflammatory cell infiltration.

FIG. 4 shows that CBD and hydroxychloroquine are effective for the treatment of mice with TNBS-induced colitis. (A) A photographic representation of normal colon tissue in sham-treated control mice; (B) A photographic representation of colon tissue from vehicle control mice, *indicates regions of inflammatory cell infiltration in submucosal edema; (C) A photographic representation of colon tissue from mice treated with CBD (1 mg/kg), *indicates regions of inflammatory cell infiltration in submucosal edema; (D) A photographic representation of colon tissue from mice treated with hydroxychloroquine (2.5 mg/kg), *indicates regions of mild abnormality, cyctic dilation and aberrant crypts; (E) A photographic representation of colon tissue from mice treated with CBD (1 mg/kg) and hydroxychloroquine (2.5 mg/kg), *indicates regions of minimal cell infiltration. All images shown at 100× magnification, and stained with hematoxylin and eosin (H&E).

FIG. 5 shows that CBD and hydroxychloroquine are effective for the treatment of rats with collagen-induced arthritis. (A) A photographic representation of normal hind paw ankle tissue in sham-treated control rats; (B) A photographic representation of hind paw ankle tissue from vehicle control rats; (C) A photographic representation of hind paw ankle tissue from rats treated with CBD (1 mg/kg); (D) A photographic representation of hind paw ankle tissue from rats treated with hydroxychloroquine (2.5 mg/kg); (E) A photographic representation of hind paw ankle tissue from rats treated with CBD (1 mg/kg) and hydroxychloroquine (2.5 mg/kg). All images shown at 50× magnification, and stained with hematoxylin and eosin (H&E).

FIG. 6 shows the dissolution profiles of 200 mg hydroxychloroquine sulfate tablets and 100 mg hydroxychloroquine sulfate tablets. A graphical representation of dissolution (%; y-axis) and sampling time (minutes, x-axis). Line indicated with diamond data points represents 200 mg hydroxychloroquine sulfate tablets. Line indicated with square data points represents 100 mg hydroxychloroquine sulfate tablets.

### DETAILED DESCRIPTION

Unless defined otherwise, all technical and scientific terms used herein have the same meaning as commonly understood by those of ordinary skill in the art to which the disclosure belongs. Any materials and method similar or equivalent to those described herein can be used to practice the present invention.

Throughout this specification, unless the context requires otherwise, the word “comprise”, or variations such as “comprises” or “comprising”, will be understood to imply the inclusion of the stated element or integer or group of elements or integers but not the exclusion of any other element or integer or group of elements or integers.

The phrase “consisting of” means including, and limited to, whatever follows the phrase “consisting of”. Thus, the phrase “consisting of” indicates that the listed elements are required or mandatory, and that no other elements may be present. The phrase “consisting essentially of” means including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein the singular forms “a”, “an” and “the” include plural aspects unless the context clearly dictates otherwise. Thus, for example, reference to “a composition” includes a single composition, as well as two or more compositions; reference to “an agent” includes one agent, as well as two or more agents; and so forth.

The term “about” will be understood by persons skilled in the art and will vary to some extent depending on the context in which it is used. If there are uses of the term that are not clear to persons skilled in the art, given the context which it is used, “about” will mean up to plus or minus 10% of the particular term.

The present disclosure is predicated, at least in part, on the inventor's surprising finding that a single dose form of cannabidiol (CBD) or a pharmaceutically acceptable salt or derivative thereof and hydroxychloroquine or a pharmaceutically acceptable salt thereof can be formulated by encapsulating the CBD or a pharmaceutically acceptable salt or derivative thereof in a first capsule and encapsulating the hydroxychloroquine or a pharmaceutically acceptable salt thereof in a second capsule, wherein the second capsule is at least partially encapsulated by the first capsule, thereby enabling the simultaneous administration of the synergistic combination of CBD and hydroxychloroquine.

Thus, in an aspect disclosed herein, there is provided a composition comprising:
a. CBD or a pharmaceutically acceptable salt or derivative thereof; and
b. a solid dosage form of hydroxychloroquine or a pharmaceutically acceptable salt thereof,
   wherein the CBD or a pharmaceutically acceptable salt or derivative thereof is encapsulated by a first capsule and the solid dosage form of hydroxychloroquine or a pharmaceutically acceptable salt thereof is encapsulated by a second capsule, wherein the second capsule is at least partially encapsulated by the first capsule.

### Compositions

In accordance with the present disclosure, the composition allows for the delivery of two synergistic active pharmaceutical ingredients (APIs) that have not previously been combined in a single dosage form. The composition described herein advantageously avoids any problems associated with the incompatibility of APIs, which may limit the ability to formulate the combination into a single dosage form or fixed dose combination (FDC).

The phrase “fixed dose combination” or “FDC” as used herein refers to a combination of two or more APIs contained in a single dosage form, such as a capsule or tablet. Accordingly, the composition described herein may also be referred to as an FDC.

The configuration of the composition described herein, that is, CBD or a pharmaceutically acceptable salt or derivative thereof encapsulated in by a first capsule, and a solid dosage form of hydroxychloroquine or a pharmaceutically acceptable salt thereof encapsulated in a second capsule, wherein the second capsule is at least partially encapsulated by the first capsule provides a physical barrier between the two APIs. As described elsewhere herein, the physical separation of the two APIs avoids any problems with incompatibility between the CBD and hydroxychloroquine, which may limit the ability to provide a synergistic FDC.

Encapsulation may be achieved using any film-forming material known in the art, illustrative examples of which include gelatin, starch, carrageenans, gums or synthetic materials such as hydroxypropyl-methylcellulose (HPMC), other hydroxyalkylated celluloses and the like. The film-forming material typically has an aqueous base and is ingestible. As used herein the term “ingestible” refers to a film-forming material that dissolves under conditions simulating the human digestive tract or water.

In an embodiment, the first capsule may comprise, consist of or substantially consist of animal or non-animal based material. Suitable non-animal material for capsules would be known to persons skilled in the art, illustrative examples of which include starch, carrageenans, gums or synthetic materials such as hydroxalkylated celluloses (e.g., hydroxypropyl methylcellulose) and polymers (e.g., synthetic polymers). Suitable animal materials for capsules would be known to persons skilled in the art, illustrative examples of which include hard gelatin capsules and soft gelatin capsules.

In an embodiment, the first capsule is a soft gelatin capsule.

Soft gelatin capsules or “softgels” generally comprise an outer shell primarily made of gelatin, a plasticizer, and water. Soft gelatin capsules are widely used in the pharmaceutical industry for oral administration or as suppositories for rectal or vaginal use. Other uses are topical and ophthalmic preparations and the like, e.g., the cosmetic industry use soft gelatin capsules as a specialized package for various types of perfumes, oils, shampoo, skin creams and the like. Soft gelatin capsules are available in a variety of sizes and shapes, e.g., tubes, ovals, oblongs, tubes and other special types of shapes such as stars. The finished soft gelatin capsules can be made in a variety of colors and opacifiers may be added to the soft gelatin capsules.

Processes for the manufacture of soft gelatin capsules and encapsulation of APIs within soft gelatin capsules would be known to persons skilled in the art, illustrative examples of which include the methods described in U.S. Pat. Nos. 9,433,584, 10,383,826 and WO 2012/017325.

In an embodiment, the soft gelatin capsule comprises a gelatin selected from the group consisting of bovine gelatin, porcine gelatin, fish gelatin and blends thereof.

It is also contemplated herein that the soft gelatin capsule may comprise one or more of starch, carrageenans, gums or synthetic materials such as hydroxalkylated celluloses.

In an embodiment, the CBD or a pharmaceutically acceptable salt or derivative thereof is solubilized in a liquid solvent. Suitable liquid solvents would be known to persons skilled in the art, illustrative examples of which include oils, alcohol (e.g., ethanol), propylene glycol and glycerol.

In an embodiment, the liquid solvent is an oil selected from the group consisting of hemp seed oil, olive oil, caprylic/capric triglyceride (MCT) oil, sunflower oil and sesame seed oil.

In a preferred embodiment, the oil is sesame seed oil.

In an embodiment, the liquid solvent further comprises one or more agents selected from the group of sweetening agents, flavoring agents, coloring agents and preserving agents. Suitable sweeteners include sucrose, lactose, glucose, aspartame or saccharin. Suitable flavoring agents include peppermint oil, oil of wintergreen, cherry, orange or raspberry flavoring. Suitable preservatives include sodium benzoate, vitamin E, alphatocopherol, ascorbic acid, methyl paraben, propyl paraben, sodium bisulphite or butylated hydroxytoluene (BHT).

In an embodiment, the liquid solvent further comprises BHT.

As described elsewhere herein, the composition beneficially separates one API (i.e., CBD) from the second API (i.e., hydroxychloroquine) by virtue of the second capsule that encapsulates the second API. In an embodiment, and without being bound by a particular theory, the separation of the first API and the second API is achieved based on the encapsulation of the second API using a material that is not solubilized by the liquid solvent in which the CBD or a pharmaceutically acceptable salt or derivative thereof has been solubilized in. For example, the CBD or a pharmaceutically acceptable salt or derivative thereof may be formulated at a pH range that prevents the second capsule from being solubilized.

Accordingly, in an embodiment, the second capsule is a capsule that is not solubilized by the liquid solvent.

The second capsule may comprise, consist of or substantially consist of animal or non-animal based material as described elsewhere herein.

In an embodiment, the second capsule is selected from the group consisting of a polymer capsule, a soft gelatin capsule, and a cellulose capsule.

In an embodiment, the second capsule is a soft gelatin capsule.

In another embodiment, the second capsule is a polymer capsule.

Suitable polymer capsules would be known to persons skilled in the art, illustrative examples of which include capsules comprising polyvinyl alcohol, hydroxypropyl methylcellulose, hydroxypropyl methylcellulose phthalate, hydroxypropyl methylcellulose acetate succinate, cellulose polymer, polyvinyl acetate, cellulose acetate phthalate, cellulose acetate trimellitate and polyvinyl acetate phthalate. In an embodiment, the polymer capsule comprises polyvinyl alcohol (e.g., Opadry® 200).

In some embodiments, the first capsule and the second capsule are the same material, e.g., soft gelatin capsules. In another embodiment, the first capsule and the second capsule are different materials, e.g., a soft gelatin first capsule and a polymer second capsule.

In another embodiment, the second capsule is an enteric capsule.

The term “enteric” as used herein means a coating material to provide slow, modified or controlled release of the solid dosage form of hydroxychloroquine or a pharmaceutically acceptable salt thereof. The enteric capsule may also be used to prevent dissolution of the solid dosage form of hydroxychloroquine or a pharmaceutically acceptable salt thereof in the gastric environment.

In an embodiment, the second capsule further comprises a coating. Suitable coatings would be known to persons skilled in the art, illustrative examples of which include sealants, glazes, polishes and shellac. The coatings contemplated herein provide an additional functional barrier to moisture and far. In an embodiment, the coating is a clear coating comprising sodium carboxymethylcellulose, maltodexrin, dextrose monohydrate and purified stearic acid (i.e., Opaglos® 2).

The phrase “solid dosage form” as used herein refers to any solid form of hydroxychloroquine or a pharmaceutically acceptable salt thereof, such as tablets, caplets, granules, nanoparticles, pellets and the like. Accordingly, in an embodiment, the solid dosage form is selected from the group consisting of a tablet, a powder, a microgranule, a nanoparticle and a pellet.

In a preferred embodiment, the solid dosage form is a tablet.

The phrase “at least partially encapsulated” refers to the complete or partial encapsulation of the solid dosage form of the hydroxychloroquine or a pharmaceutically acceptable salt thereof by the first capsule. Suitable configurations of the first capsule and the second capsule would be determined by persons skilled in the art by reference to, e.g., the manufacturing process, encapsulation materials and the formulation of the CBD and solid dosage form of the hydroxychloroquine.

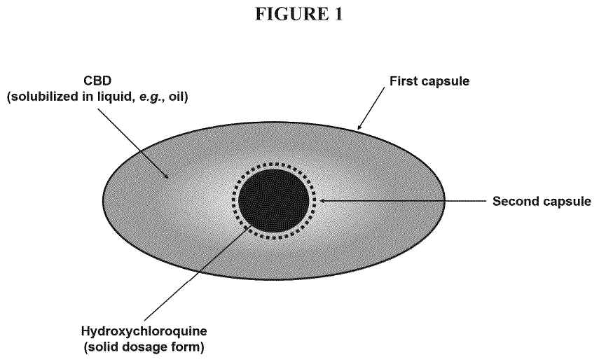

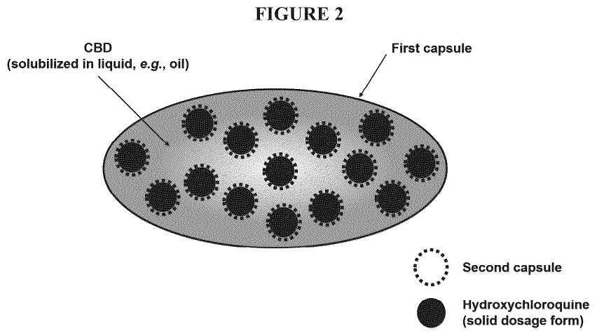

In an embodiment, the second capsule is completely incorporated within the first capsule. For example, the second capsule may be completely incorporated in accordance with the schematic representation of the composition shown in FIGS. 1 and 2.

In a preferred embodiment, the composition is formulated for oral administration.

Compositions for oral administration may contain one or more agents selected from the group of sweetening agents, flavoring agents, coloring agents and preserving agents in order to produce pharmaceutically elegant and palatable preparations. Suitable sweeteners include sucrose, lactose, glucose, aspartame or saccharin. Suitable disintegrating agents include corn starch, methylcellulose, polyvinylpyrrolidone, xanthan gum, bentonite, alginic acid or agar. Suitable flavoring agents include peppermint oil, oil of wintergreen, cherry, orange or raspberry flavoring. Suitable preservatives include sodium benzoate, vitamin E, alphatocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

In an embodiment, the composition further comprises one or more pharmaceutically acceptable carriers, diluents or excipients.

Suitable pharmaceutically acceptable carriers, diluents or excipients would be known to persons skilled in the art, illustrative examples of which include inert diluents (e.g., calcium carbonate, lactose, calcium phosphate or sodium phosphate), granulating and disintegrating agents (e.g., corn starch or alginic acid), binding agents (e.g., starch, gelatin or acacia), lubricating agents (e.g., magnesium stearate, stearic acid or talc) and material to delay disintegration and absorption in the gastrointestinal tract and thereby provide a sustained action over a longer period (e.g., glyceryl monostearate or glyceryl distearate).

Compositions disclosed herein may be prepared according to conventional methods well known in the pharmaceutical and nutraceutical industries, such as those described in Remington's Pharmaceutical Handbook (Mack Publishing Co., NY, USA).

Oral administration of CBD has been demonstrated to be an effective administration route (reviewed by Millar et al., 2018, Frontiers in Pharmacology, 9: 1365). Similarly, oral administration of hydroxychloroquine has also been demonstrated to be effective for absorption with high bioavailability (Tett et al., 1989, British Journal of Clinical Pharmacology, 27: 771-779).

### Cannabidiol

“Cannabidiol” or “CBD” is a cannabinoid produced by plants of the genus Cannabis. CBD has antagonist activity on agonists of the CB1 and CB2 receptors and acts as an inverse agonist of the CB1 and CB2 receptors.

CBD is synthesized in cannabis plants as cannabidiolic acid (CBDA), which decarboxylates to CBD (Table 1). While some decarboxylation may occur in the plant, decarboxylation typically occurs post-harvest and is increased by exposing plant material to heat (Sanchez and Verpoote, 2008, Plant Cell Physiology, 49(12): 1767-82). Decarboxylation is usually achieved by drying and/or heating the plant material. Persons skilled in the art would be familiar with methods by which decarboxylation of CBDA can be promoted, illustrative examples of which include air-drying, combustion, vaporization, curing, heating and baking. The decarboxylated CBD will typically bind to and/or stimulate, directly or indirectly, cannabinoid receptors including CB1 and/or CB2.

CBD may be extracted from any suitable plant parts including leaves, flowers or stems and may be produced by any suitable means known to those skilled in the art. For example, CBD extracts may be produced by extraction with supercritical or subcritical CO₂, or by volatilization of plant material with a heated gas. Illustrative examples of methods used the extract CBD and other cannabinoids from plant material include the methods described in U.S. patent Ser. No. 10/189,762 and WO 2004/016277.

In an embodiment, the CBD described herein is a synthetic compound.

Synthesized CBD is particularly useful for pharmaceutical development it is largely free from contaminants. A number of methods for the synthesis of CBD are known in the art, illustrative examples of which include methods for the synthesis of CBD as described in U.S. Pat. No. 10,059,682.

In an embodiment, the synthesized CBD comprises, consists or consists essentially of the (−)-CBD enantiomer.

The present disclosure further contemplates the use of pharmaceutically acceptable salts or derivatives of CBD. Suitable pharmaceutically acceptable salts or derivatives of CBD would be known to persons skilled in the art, illustrative examples of which include dihydro- and tetrahydro-cannabidiol derivatives H2-CBD and H4-CBD, the (−)- and (+)-dihydro-7-hydroxy-CBD enantiomers and 1′,1′-dimethylheptyl-CBD derivatives.

In an embodiment, the composition comprises at least about 25 mg CBD or a pharmaceutically acceptable salt or derivative thereof (e.g., 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 131 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, 400 mg, 401 mg, 402 mg, 403 mg, 404 mg, 405 mg, 406 mg, 407 mg, 408 mg, 409 mg, 410 mg, 411 mg, 412 mg, 413 mg, 414 mg, 415 mg, 416 mg, 417 mg, 418 mg, 419 mg, 420 mg, 421 mg, 422 mg, 423 mg, 424 mg, 425 mg, 426 mg, 427 mg, 428 mg, 429 mg, 430 mg, 431 mg, 432 mg, 433 mg, 434 mg, 435 mg, 436 mg, 437 mg, 438 mg, 439 mg, 440 mg, 441 mg, 442 mg, 443 mg, 444 mg, 445 mg, 446 mg, 447 mg, 448 mg, 449 mg, 450 mg, 451 mg, 452 mg, 453 mg, 454 mg, 455 mg, 456 mg, 457 mg, 458 mg, 459 mg, 460 mg, 461 mg, 462 mg, 463 mg, 464 mg, 465 mg, 466 mg, 467 mg, 468 mg, 469 mg, 470 mg, 471 mg, 472 mg, 473 mg, 474 mg, 475 mg, 476 mg, 477 mg, 478 mg, 479 mg, 480 mg, 481 mg, 482 mg, 483 mg, 484 mg, 485 mg, 486 mg, 487 mg, 488 mg, 489 mg, 490 mg, 491 mg, 492 mg, 493 mg, 494 mg, 495 mg, 496 mg, 497 mg, 498 mg, 499 mg, 500 mg, 501 mg, 502 mg, 503 mg, 504 mg, 505 mg, 506 mg, 507 mg, 508 mg, 509 mg, 510 mg, 511 mg, 512 mg, 513 mg, 514 mg, 515 mg, 516 mg, 517 mg, 518 mg, 519 mg, 520 mg, 521 mg, 522 mg, 523 mg, 524 mg, 525 mg, 526 mg, 527 mg, 528 mg, 529 mg, 530 mg, 531 mg, 532 mg, 533 mg, 534 mg, 535 mg, 536 mg, 537 mg, 538 mg, 539 mg, 540 mg, 541 mg, 542 mg, 543 mg, 544 mg, 545 mg, 546 mg, 547 mg, 548 mg, 549 mg, 550 mg, 551 mg, 552 mg, 553 mg, 554 mg, 555 mg, 556 mg, 557 mg, 558 mg, 559 mg, 560 mg, 561 mg, 562 mg, 563 mg, 564 mg, 565 mg, 566 mg, 567 mg, 568 mg, 569 mg, 570 mg, 571 mg, 572 mg, 573 mg, 574 mg, 575 mg, 576 mg, 577 mg, 578 mg, 579 mg, 580 mg, 581 mg, 582 mg, 583 mg, 584 mg, 585 mg, 586 mg, 587 mg, 588 mg, 589 mg, 590 mg, 591 mg, 592 mg, 593 mg, 594 mg, 595 mg, 596 mg, 597 mg, 598 mg, 599 mg, 600 mg, 601 mg, 602 mg, 603 mg, 604 mg, 605 mg, 606 mg, 607 mg, 608 mg, 609 mg, 610 mg, 611 mg, 612 mg, 613 mg, 614 mg, 615 mg, 616 mg, 617 mg, 618 mg, 619 mg, 620 mg, 621 mg, 622 mg, 623 mg, 624 mg, 625 mg, 626 mg, 627 mg, 628 mg, 629 mg, 630 mg, 631 mg, 632 mg, 633 mg, 634 mg, 635 mg, 636 mg, 637 mg, 638 mg, 639 mg, 640 mg, 641 mg, 642 mg, 643 mg, 644 mg, 645 mg, 646 mg, 647 mg, 648 mg, 649 mg, 650 mg, 651 mg, 652 mg, 653 mg, 654 mg, 655 mg, 656 mg, 657 mg, 658 mg, 659 mg, 660 mg, 661 mg, 662 mg, 663 mg, 664 mg, 665 mg, 666 mg, 667 mg, 668 mg, 669 mg, 670 mg, 671 mg, 672 mg, 673 mg, 674 mg, 675 mg, 676 mg, 677 mg, 678 mg, 679 mg, 680 mg, 681 mg, 682 mg, 683 mg, 684 mg, 685 mg, 686 mg, 687 mg, 688 mg, 689 mg, 690 mg, 691 mg, 692 mg, 693 mg, 694 mg, 695 mg, 696 mg, 697 mg, 698 mg, 699 mg, 700 mg, 701 mg, 702 mg, 703 mg, 704 mg, 705 mg, 706 mg, 707 mg, 708 mg, 709 mg, 710 mg, 711 mg, 712 mg, 713 mg, 714 mg, 715 mg, 716 mg, 717 mg, 718 mg, 719 mg, 720 mg, 721 mg, 722 mg, 723 mg, 724 mg, 725 mg, 726 mg, 727 mg, 728 mg, 729 mg, 730 mg, 731 mg, 732 mg, 733 mg, 734 mg, 735 mg, 736 mg, 737 mg, 738 mg, 739 mg, 740 mg, 741 mg, 742 mg, 743 mg, 744 mg, 745 mg, 746 mg, 747 mg, 748 mg, 749 mg, 750 mg, 751 mg, 752 mg, 753 mg, 754 mg, 755 mg, 756 mg, 757 mg, 758 mg, 759 mg, 760 mg, 761 mg, 762 mg, 763 mg, 764 mg, 765 mg, 766 mg, 767 mg, 768 mg, 769 mg, 770 mg, 771 mg, 772 mg, 773 mg, 774 mg, 775 mg, 776 mg, 777 mg, 778 mg, 779 mg, 780 mg, 781 mg, 782 mg, 783 mg, 784 mg, 785 mg, 786 mg, 787 mg, 788 mg, 789 mg, 790 mg, 791 mg, 792 mg, 793 mg, 794 mg, 795 mg, 796 mg, 797 mg, 798 mg, 799 mg, 800 mg, 801 mg, 802 mg, 803 mg, 804 mg, 805 mg, 806 mg, 807 mg, 808 mg, 809 mg, 810 mg, 811 mg, 812 mg, 813 mg, 814 mg, 815 mg, 816 mg, 817 mg, 818 mg, 819 mg, 820 mg, 821 mg, 822 mg, 823 mg, 824 mg, 825 mg, 826 mg, 827 mg, 828 mg, 829 mg, 830 mg, 831 mg, 832 mg, 833 mg, 834 mg, 835 mg, 836 mg, 837 mg, 838 mg, 839 mg, 840 mg, 841 mg, 842 mg, 843 mg, 844 mg, 845 mg, 846 mg, 847 mg, 848 mg, 849 mg, 850 mg, 851 mg, 852 mg, 853 mg, 854 mg, 855 mg, 856 mg, 857 mg, 858 mg, 859 mg, 860 mg, 861 mg, 862 mg, 863 mg, 864 mg, 865 mg, 866 mg, 867 mg, 868 mg, 869 mg, 870 mg, 871 mg, 872 mg, 873 mg, 874 mg, 875 mg, 876 mg, 877 mg, 878 mg, 879 mg, 880 mg, 881 mg, 882 mg, 883 mg, 884 mg, 885 mg, 886 mg, 887 mg, 888 mg, 889 mg, 890 mg, 891 mg, 892 mg, 893 mg, 894 mg, 895 mg, 896 mg, 897 mg, 898 mg, 899 mg, 900 mg, 901 mg, 902 mg, 903 mg, 904 mg, 905 mg, 906 mg, 907 mg, 908 mg, 909 mg, 910 mg, 911 mg, 912 mg, 913 mg, 914 mg, 915 mg, 916 mg, 917 mg, 918 mg, 919 mg, 920 mg, 921 mg, 922 mg, 923 mg, 924 mg, 925 mg, 926 mg, 927 mg, 928 mg, 929 mg, 930 mg, 931 mg, 932 mg, 933 mg, 934 mg, 935 mg, 936 mg, 937 mg, 938 mg, 939 mg, 940 mg, 941 mg, 942 mg, 943 mg, 944 mg, 945 mg, 946 mg, 947 mg, 948 mg, 949 mg, 950 mg, 951 mg, 952 mg, 953 mg, 954 mg, 955 mg, 956 mg, 957 mg, 958 mg, 959 mg, 960 mg, 961 mg, 962 mg, 963 mg, 964 mg, 965 mg, 966 mg, 967 mg, 968 mg, 969 mg, 970 mg, 971 mg, 972 mg, 973 mg, 974 mg, 975 mg, 976 mg, 977 mg, 978 mg, 979 mg, 980 mg, 981 mg, 982 mg, 983 mg, 984 mg, 985 mg, 986 mg, 987 mg, 988 mg, 989 mg, 990 mg, 991 mg, 992 mg, 993 mg, 994 mg, 995 mg, 996 mg, 997 mg, 998 mg, 999 mg, 1000 mg, 1100 mg, 1101 mg, 1102 mg, 1103 mg, 1104 mg, 1105 mg, 1106 mg, 1107 mg, 1108 mg, 1109 mg, 1110 mg, 1111 mg, 1112 mg, 1113 mg, 1114 mg, 1115 mg, 1116 mg, 1117 mg, 1118 mg, 1119 mg, 1120 mg, 1121 mg, 1122 mg, 1123 mg, 1124 mg, 1125 mg, 1126 mg, 1127 mg, 1128 mg, 1129 mg, 1130 mg, 1131 mg, 1132 mg, 1133 mg, 1134 mg, 1135 mg, 1136 mg, 1137 mg, 1138 mg, 1139 mg, 1140 mg, 1141 mg, 1142 mg, 1143 mg, 1144 mg, 1145 mg, 1146 mg, 1147 mg, 1148 mg, 1149 mg, 1150 mg, 1151 mg, 1152 mg, 1153 mg, 1154 mg, 1155 mg, 1156 mg, 1157 mg, 1158 mg, 1159 mg, 1160 mg, 1161 mg, 1162 mg, 1163 mg, 1164 mg, 1165 mg, 1166 mg, 1167 mg, 1168 mg, 1169 mg, 1170 mg, 1171 mg, 1172 mg, 1173 mg, 1174 mg, 1175 mg, 1176 mg, 1177 mg, 1178 mg, 1179 mg, 1180 mg, 1181 mg, 1182 mg, 1183 mg, 1184 mg, 1185 mg, 1186 mg, 1187 mg, 1188 mg, 1189 mg, 1190 mg, 1191 mg, 1192 mg, 1193 mg, 1194 mg, 1195 mg, 1196 mg, 1197 mg, 1198 mg, 1199 mg, 1200 mg, 1201 mg, 1202 mg, 1203 mg, 1204 mg, 1205 mg, 1206 mg, 1207 mg, 1208 mg, 1209 mg, 1210 mg, 1211 mg, 1212 mg, 1213 mg, 1214 mg, 1215 mg, 1216 mg, 1217 mg, 1218 mg, 1219 mg, 1220 mg, 1221 mg, 1222 mg, 1223 mg, 1224 mg, 1225 mg, 1226 mg, 1227 mg, 1228 mg, 1229 mg, 1230 mg, 1231 mg, 1232 mg, 1233 mg, 1234 mg, 1235 mg, 1236 mg, 1237 mg, 1238 mg, 239 mg, 1240 mg, 1241 mg, 1242 mg, 1243 mg, 1244 mg, 1245 mg, 1246 mg, 1247 mg, 1248 mg, 1249 mg, 1250 mg, 1251 mg, 1252 mg, 1253 mg, 1254 mg, 1255 mg, 1256 mg, 1257 mg, 1258 mg, 1259 mg, 1260 mg, 1261 mg, 1262 mg, 1263 mg, 1264 mg, 1265 mg, 1266 mg, 1267 mg, 1268 mg, 1269 mg, 1270 mg, 1271 mg, 1272 mg, 1273 mg, 1274 mg, 1275 mg, 1276 mg, 1277 mg, 1278 mg, 1279 mg, 1280 mg, 1281 mg, 1282 mg, 1283 mg, 1284 mg, 1285 mg, 1286 mg, 1287 mg, 1288 mg, 1289 mg, 1290 mg, 1291 mg, 1292 mg, 1293 mg, 1294 mg, 1295 mg, 1296 mg, 1297 mg, 1298 mg, 1299 mg, 1300 mg, 1301 mg, 1302 mg, 303 mg, 1304 mg, 1305 mg, 1306 mg, 1307 mg, 1308 mg, 1309 mg, 1310 mg, 1311 mg, 1312 mg, 1313 mg, 1314 mg, 1315 mg, 1316 mg, 1317 mg, 1318 mg, 1319 mg, 1320 mg, 1321 mg, 1322 mg, 1323 mg, 1324 mg, 1325 mg, 1326 mg, 1327 mg, 1328 mg, 1329 mg, 1330 mg, 1331 mg, 1332 mg, 1333 mg, 1334 mg, 1335 mg, 1336 mg, 1337 mg, 1338 mg, 1339 mg, 1340 mg, 1341 mg, 1342 mg, 1343 mg, 1344 mg, 1345 mg, 1346 mg, 1347 mg, 1348 mg, 1349 mg, 1350 mg, 1351 mg, 1352 mg, 1353 mg, 1354 mg, 1355 mg, 1356 mg, 1357 mg, 1358 mg, 1359 mg, 1360 mg, 1361 mg, 1362 mg, 1363 mg, 1364 mg, 1365 mg, 1366 mg, 1367 mg, 1368 mg, 1369 mg, 1370 mg, 1371 mg, 1372 mg, 1373 mg, 1374 mg, 1375 mg, 1376 mg, 1377 mg, 1378 mg, 1379 mg, 1380 mg, 1381 mg, 1382 mg, 1383 mg, 1384 mg, 1385 mg, 1386 mg, 1387 mg, 1388 mg, 1389 mg, 1390 mg, 1391 mg, 1392 mg, 1393 mg, 1394 mg, 1395 mg, 1396 mg, 1397 mg, 1398 mg, 1399 mg, 1400 mg, 1401 mg, 1402 mg, 1403 mg, 1404 mg, 1405 mg, 1406 mg, 1407 mg, 1408 mg, 1409 mg, 1410 mg, 1411 mg, 1412 mg, 1413 mg, 1414 mg, 1415 mg, 1416 mg, 1417 mg, 1418 mg, 1419 mg, 1420 mg, 1421 mg, 1422 mg, 1423 mg, 1424 mg, 1425 mg, 1426 mg, 1427 mg, 1428 mg, 1429 mg, 1430 mg, 1431 mg, 1432 mg, 1433 mg, 1434 mg, 1435 mg, 1436 mg, 1437 mg, 1438 mg, 1439 mg, 1440 mg, 1441 mg, 1442 mg, 1443 mg, 1444 mg, 1445 mg, 1446 mg, 1447 mg, 1448 mg, 1449 mg, 1450 mg, 1451 mg, 1452 mg, 1453 mg, 1454 mg, 1455 mg, 1456 mg, 1457 mg, 1458 mg, 1459 mg, 1460 mg, 1461 mg, 1462 mg, 1463 mg, 1464 mg, 1465 mg, 1466 mg, 1467 mg, 1468 mg, 1469 mg, 1470 mg, 1471 mg, 1472 mg, 1473 mg, 1474 mg, 1475 mg, 1476 mg, 1477 mg, 1478 mg, 1479 mg, 1480 mg, 1481 mg, 1482 mg, 1483 mg, 1484 mg, 1485 mg, 1486 mg, 1487 mg, 1488 mg, 1489 mg, 1490 mg, 1491 mg, 1492 mg, 1493 mg, 1494 mg, 1495 mg, 1496 mg, 1497 mg, 1498 mg, 1499 mg, or 1500 mg).

In an embodiment, the composition comprises from about 25 mg to about 100 mg CBD or a pharmaceutically acceptable salt or derivative thereof, preferably about 25 mg, preferably about 26 mg, preferably about 27 mg, preferably about 28 mg, preferably about 29 mg, preferably about 30 mg, preferably about 31 mg, preferably about 32 mg, preferably about 33 mg, preferably about 34 mg, preferably about 35 mg, preferably about 36 mg, preferably about 37 mg, preferably about 38 mg, preferably about 39 mg, preferably about 40 mg, preferably about 41 mg, preferably about 42 mg, preferably about 43 mg, preferably about 44 mg, preferably about 45 mg, preferably about 46 mg, preferably about 47 mg, preferably about 48 mg, preferably about 49 mg, preferably about 50 mg, preferably about 51 mg, preferably about 52 mg, preferably about 53 mg, preferably about 54 mg, preferably about 55 mg, preferably about 56 mg, preferably about 57 mg, preferably about 58 mg, preferably about 59 mg, preferably about 60 mg, preferably about 61 mg, preferably about 62 mg, preferably about 63 mg, preferably about 64 mg, preferably about 65 mg, preferably about 66 mg, preferably about 67 mg, preferably about 68 mg, preferably about 69 mg, preferably about 70 mg, preferably about 71 mg, preferably about 72 mg, preferably about 73 mg, preferably about 74 mg, preferably about 75 mg, preferably about 76 mg, preferably about 77 mg, preferably about 78 mg, preferably about 79 mg, preferably about 80 mg, preferably about 81 mg, preferably about 82 mg, preferably about 83 mg, preferably about 84 mg, preferably about 85 mg, preferably about 86 mg, preferably about 87 mg, preferably about 88 mg, preferably about 89 mg, preferably about 90 mg, preferably about 91 mg, preferably about 92 mg, preferably about 93 mg, preferably about 94 mg, preferably about 95 mg, preferably about 96 mg, preferably about 97 mg, preferably about 98 mg, preferably about 99 mg, or more preferably about 100 mg.

In a preferred embodiment, the composition comprises about 75 mg CBD or a pharmaceutically acceptable salt or derivative thereof.

### Hydroxychloroquine

“Hydroxychloroquine” is a chemical derivative of chloroquine, which features a hydroxyethyl group instead of an ethyl group. Hydroxychloroquine, commonly referred to by the trade name “Plaquenil®” is known to be effective for the treatment of malaria, and has shown efficacy for the treatment of systemic lupus erythematosus, rheumatoid arthritis and Sjögren's Syndrome. Functionally, hydroxychloroquine increases lysosomal pH in antigen presenting cells, and has been demonstrated to inhibit or block the activation of toll-like receptors on plasmacytoid dendritic cells.

As used herein, the term “hydroxychloroquine” includes the racemic hydroxychloroquine, which is 2-[[4-[(7-chloro-4-quinolinyl)amino]pentyl]-thylamino]ethanol as disclosed in U.S. Pat. No. 2,546,658, or any of the single enantiomers “(S)-(+) hydroxychloroquine” or “(R)-(−) hydroxychloroquine” as disclosed in U.S. Pat. No. 5,314,894. This term may relate either to the free form of hydroxychloroquine or to any pharmaceutically acceptable salt thereof, such as hydroxychloroquine sulfate.

The present disclosure further contemplates the use of pharmaceutically acceptable salts of hydroxychloroquine. Suitable pharmaceutically acceptable salts would be known to person skilled in the art, illustrative examples of which include salts or esters prepared from pharmaceutically acceptable non-toxic bases or acids, including inorganic bases or acids and organic bases or acids, which would be well known to person skilled in the art.

In an embodiment, the pharmaceutically acceptable salt is hydroxychloroquine sulfate.

In an embodiment, the composition comprises at least about 10 mg hydroxychloroquine or a pharmaceutically acceptable salt thereof (e.g., 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 131 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, 400 mg, 401 mg, 402 mg, 403 mg, 404 mg, 405 mg, 406 mg, 407 mg, 408 mg, 409 mg, 410 mg, 411 mg, 412 mg, 413 mg, 414 mg, 415 mg, 416 mg, 417 mg, 418 mg, 419 mg, 420 mg, 421 mg, 422 mg, 423 mg, 424 mg, 425 mg, 426 mg, 427 mg, 428 mg, 429 mg, 430 mg, 431 mg, 432 mg, 433 mg, 434 mg, 435 mg, 436 mg, 437 mg, 438 mg, 439 mg, 440 mg, 441 mg, 442 mg, 443 mg, 444 mg, 445 mg, 446 mg, 447 mg, 448 mg, 449 mg, 450 mg, 451 mg, 452 mg, 453 mg, 454 mg, 455 mg, 456 mg, 457 mg, 458 mg, 459 mg, 460 mg, 461 mg, 462 mg, 463 mg, 464 mg, 465 mg, 466 mg, 467 mg, 468 mg, 469 mg, 470 mg, 471 mg, 472 mg, 473 mg, 474 mg, 475 mg, 476 mg, 477 mg, 478 mg, 479 mg, 480 mg, 481 mg, 482 mg, 483 mg, 484 mg, 485 mg, 486 mg, 487 mg, 488 mg, 489 mg, 490 mg, 491 mg, 492 mg, 493 mg, 494 mg, 495 mg, 496 mg, 497 mg, 498 mg, 499 mg, 500 mg, 501 mg, 502 mg, 503 mg, 504 mg, 505 mg, 506 mg, 507 mg, 508 mg, 509 mg, 510 mg, 511 mg, 512 mg, 513 mg, 514 mg, 515 mg, 516 mg, 517 mg, 518 mg, 519 mg, 520 mg, 521 mg, 522 mg, 523 mg, 524 mg, 525 mg, 526 mg, 527 mg, 528 mg, 529 mg, 530 mg, 531 mg, 532 mg, 533 mg, 534 mg, 535 mg, 536 mg, 537 mg, 538 mg, 539 mg, 540 mg, 541 mg, 542 mg, 543 mg, 544 mg, 545 mg, 546 mg, 547 mg, 548 mg, 549 mg, 550 mg, 551 mg, 552 mg, 553 mg, 554 mg, 555 mg, 556 mg, 557 mg, 558 mg, 559 mg, 560 mg, 561 mg, 562 mg, 563 mg, 564 mg, 565 mg, 566 mg, 567 mg, 568 mg, 569 mg, 570 mg, 571 mg, 572 mg, 573 mg, 574 mg, 575 mg, 576 mg, 577 mg, 578 mg, 579 mg, 580 mg, 581 mg, 582 mg, 583 mg, 584 mg, 585 mg, 586 mg, 587 mg, 588 mg, 589 mg, 590 mg, 591 mg, 592 mg, 593 mg, 594 mg, 595 mg, 596 mg, 597 mg, 598 mg, 599 mg, 600 mg, 601 mg, 602 mg, 603 mg, 604 mg, 605 mg, 606 mg, 607 mg, 608 mg, 609 mg, 610 mg, 611 mg, 612 mg, 613 mg, 614 mg, 615 mg, 616 mg, 617 mg, 618 mg, 619 mg, 620 mg, 621 mg, 622 mg, 623 mg, 624 mg, 625 mg, 626 mg, 627 mg, 628 mg, 629 mg, 630 mg, 631 mg, 632 mg, 633 mg, 634 mg, 635 mg, 636 mg, 637 mg, 638 mg, 639 mg, 640 mg, 641 mg, 642 mg, 643 mg, 644 mg, 645 mg, 646 mg, 647 mg, 648 mg, 649 mg, 650 mg, 651 mg, 652 mg, 653 mg, 654 mg, 655 mg, 656 mg, 657 mg, 658 mg, 659 mg, 660 mg, 661 mg, 662 mg, 663 mg, 664 mg, 665 mg, 666 mg, 667 mg, 668 mg, 669 mg, 670 mg, 671 mg, 672 mg, 673 mg, 674 mg, 675 mg, 676 mg, 677 mg, 678 mg, 679 mg, 680 mg, 681 mg, 682 mg, 683 mg, 684 mg, 685 mg, 686 mg, 687 mg, 688 mg, 689 mg, 690 mg, 691 mg, 692 mg, 693 mg, 694 mg, 695 mg, 696 mg, 697 mg, 698 mg, 699 mg, 700 mg, 701 mg, 702 mg, 703 mg, 704 mg, 705 mg, 706 mg, 707 mg, 708 mg, 709 mg, 710 mg, 711 mg, 712 mg, 713 mg, 714 mg, 715 mg, 716 mg, 717 mg, 718 mg, 719 mg, 720 mg, 721 mg, 722 mg, 723 mg, 724 mg, 725 mg, 726 mg, 727 mg, 728 mg, 729 mg, 730 mg, 731 mg, 732 mg, 733 mg, 734 mg, 735 mg, 736 mg, 737 mg, 738 mg, 739 mg, 740 mg, 741 mg, 742 mg, 743 mg, 744 mg, 745 mg, 746 mg, 747 mg, 748 mg, 749 mg, 750 mg, 751 mg, 752 mg, 753 mg, 754 mg, 755 mg, 756 mg, 757 mg, 758 mg, 759 mg, 760 mg, 761 mg, 762 mg, 763 mg, 764 mg, 765 mg, 766 mg, 767 mg, 768 mg, 769 mg, 770 mg, 771 mg, 772 mg, 773 mg, 774 mg, 775 mg, 776 mg, 777 mg, 778 mg, 779 mg, 780 mg, 781 mg, 782 mg, 783 mg, 784 mg, 785 mg, 786 mg, 787 mg, 788 mg, 789 mg, 790 mg, 791 mg, 792 mg, 793 mg, 794 mg, 795 mg, 796 mg, 797 mg, 798 mg, 799 mg, 800 mg, 801 mg, 802 mg, 803 mg, 804 mg, 805 mg, 806 mg, 807 mg, 808 mg, 809 mg, 810 mg, 811 mg, 812 mg, 813 mg, 814 mg, 815 mg, 816 mg, 817 mg, 818 mg, 819 mg, 820 mg, 821 mg, 822 mg, 823 mg, 824 mg, 825 mg, 826 mg, 827 mg, 828 mg, 829 mg, 830 mg, 831 mg, 832 mg, 833 mg, 834 mg, 835 mg, 836 mg, 837 mg, 838 mg, 839 mg, 840 mg, 841 mg, 842 mg, 843 mg, 844 mg, 845 mg, 846 mg, 847 mg, 848 mg, 849 mg, 850 mg, 851 mg, 852 mg, 853 mg, 854 mg, 855 mg, 856 mg, 857 mg, 858 mg, 859 mg, 860 mg, 861 mg, 862 mg, 863 mg, 864 mg, 865 mg, 866 mg, 867 mg, 868 mg, 869 mg, 870 mg, 871 mg, 872 mg, 873 mg, 874 mg, 875 mg, 876 mg, 877 mg, 878 mg, 879 mg, 880 mg, 881 mg, 882 mg, 883 mg, 884 mg, 885 mg, 886 mg, 887 mg, 888 mg, 889 mg, 890 mg, 891 mg, 892 mg, 893 mg, 894 mg, 895 mg, 896 mg, 897 mg, 898 mg, 899 mg, 900 mg, 901 mg, 902 mg, 903 mg, 904 mg, 905 mg, 906 mg, 907 mg, 908 mg, 909 mg, 910 mg, 911 mg, 912 mg, 913 mg, 914 mg, 915 mg, 916 mg, 917 mg, 918 mg, 919 mg, 920 mg, 921 mg, 922 mg, 923 mg, 924 mg, 925 mg, 926 mg, 927 mg, 928 mg, 929 mg, 930 mg, 931 mg, 932 mg, 933 mg, 934 mg, 935 mg, 936 mg, 937 mg, 938 mg, 939 mg, 940 mg, 941 mg, 942 mg, 943 mg, 944 mg, 945 mg, 946 mg, 947 mg, 948 mg, 949 mg, 950 mg, 951 mg, 952 mg, 953 mg, 954 mg, 955 mg, 956 mg, 957 mg, 958 mg, 959 mg, 960 mg, 961 mg, 962 mg, 963 mg, 964 mg, 965 mg, 966 mg, 967 mg, 968 mg, 969 mg, 970 mg, 971 mg, 972 mg, 973 mg, 974 mg, 975 mg, 976 mg, 977 mg, 978 mg, 979 mg, 980 mg, 981 mg, 982 mg, 983 mg, 984 mg, 985 mg, 986 mg, 987 mg, 988 mg, 989 mg, 990 mg, 991 mg, 992 mg, 993 mg, 994 mg, 995 mg, 996 mg, 997 mg, 998 mg, 999 mg, or 1000 mg).

In an embodiment, the composition comprises from about 10 mg to about 200 mg hydroxychloroquine or a pharmaceutically acceptable salt thereof, preferably about 10 mg, preferably about 11 mg, preferably about 12 mg, preferably about 13 mg, preferably about 14 mg, preferably about 15 mg, preferably about 16 mg, preferably about 17 mg, preferably about 18 mg, preferably about 19 mg, preferably about 20 mg, preferably about 21 mg, preferably about 22 mg, preferably about 23 mg, preferably about 24 mg, preferably about 25 mg, preferably about 26 mg, preferably about 27 mg, preferably about 28 mg, preferably about 29 mg, preferably about 30 mg, preferably about 31 mg, preferably about 32 mg, preferably about 33 mg, preferably about 34 mg, preferably about 35 mg, preferably about 36 mg, preferably about 37 mg, preferably about 38 mg, preferably about 39 mg, preferably about 40 mg, preferably about 41 mg, preferably about 42 mg, preferably about 43 mg, preferably about 44 mg, preferably about 45 mg, preferably about 46 mg, preferably about 47 mg, preferably about 48 mg, preferably about 49 mg, preferably about 50 mg, preferably about 51 mg, preferably about 52 mg, preferably about 53 mg, preferably about 54 mg, preferably about 55 mg, preferably about 56 mg, preferably about 57 mg, preferably about 58 mg, preferably about 59 mg, preferably about 60 mg, preferably about 61 mg, preferably about 62 mg, preferably about 63 mg, preferably about 64 mg, preferably about 65 mg, preferably about 66 mg, preferably about 67 mg, preferably about 68 mg, preferably about 69 mg, preferably about 70 mg, preferably about 71 mg, preferably about 72 mg, preferably about 73 mg, preferably about 74 mg, preferably about 75 mg, preferably about 76 mg, preferably about 77 mg, preferably about 78 mg, preferably about 79 mg, preferably about 80 mg, preferably about 81 mg, preferably about 82 mg, preferably about 83 mg, preferably about 84 mg, preferably about 85 mg, preferably about 86 mg, preferably about 87 mg, preferably about 88 mg, preferably about 89 mg, preferably about 90 mg, preferably about 91 mg, preferably about 92 mg, preferably about 93 mg, preferably about 94 mg, preferably about 95 mg, preferably about 96 mg, preferably about 97 mg, preferably about 98 mg, preferably about 99 mg, preferably about 100 mg, preferably about 101 mg, preferably about 102 mg, preferably about 103 mg, preferably about 104 mg, preferably about 105 mg, preferably about 106 mg, preferably about 107 mg, preferably about 108 mg, preferably about 109 mg, preferably about 110 mg, preferably about 111 mg, preferably about 112 mg, preferably about 113 mg, preferably about 114 mg, preferably about 115 mg, preferably about 116 mg, preferably about 117 mg, preferably about 118 mg, preferably about 119 mg, preferably about 120 mg, preferably about 121 mg, preferably about 122 mg, preferably about 123 mg, preferably about 124 mg, preferably about 125 mg, preferably about 126 mg, preferably about 127 mg, preferably about 128 mg, preferably about 129 mg, preferably about 130 mg, preferably about 131 mg, preferably about 132 mg, preferably about 131 mg, preferably about 134 mg, preferably about 135 mg, preferably about 136 mg, preferably about 137 mg, preferably about 138 mg, preferably about 139 mg, preferably about 140 mg, preferably about 141 mg, preferably about 142 mg, preferably about 143 mg, preferably about 144 mg, preferably about 145 mg, preferably about 146 mg, preferably about 147 mg, preferably about 148 mg, preferably about 149 mg, preferably about 150 mg, preferably about 151 mg, preferably about 152 mg, preferably about 153 mg, preferably about 154 mg, preferably about 155 mg, preferably about 156 mg, preferably about 157 mg, preferably about 158 mg, preferably about 159 mg, preferably about 160 mg, preferably about 161 mg, preferably about 162 mg, preferably about 163 mg, preferably about 164 mg, preferably about 165 mg, preferably about 166 mg, preferably about 167 mg, preferably about 168 mg, preferably about 169 mg, preferably about 170 mg, preferably about 171 mg, preferably about 172 mg, preferably about 173 mg, preferably about 174 mg, preferably about 175 mg, preferably about 176 mg, preferably about 177 mg, preferably about 178 mg, preferably about 179 mg, preferably about 180 mg, preferably about 181 mg, preferably about 182 mg, preferably about 183 mg, preferably about 184 mg, preferably about 185 mg, preferably about 186 mg, preferably about 187 mg, preferably about 188 mg, preferably about 189 mg, preferably about 190 mg, preferably about 191 mg, preferably about 192 mg, preferably about 193 mg, preferably about 194 mg, preferably about 195 mg, preferably about 196 mg, preferably about 197 mg, preferably about 198 mg, preferably about 199 mg, or more preferably about 200 mg.

In a preferred embodiment, the composition comprises about 20 mg hydroxychloroquine or a pharmaceutically acceptable salt thereof.

In another preferred embodiment, the composition comprises about 100 mg hydroxychloroquine or a pharmaceutically acceptable salt thereof.

In an embodiment, the composition defined herein is for use in the treatment of an inflammatory condition.

### Inflammatory Conditions

As used herein, the term “inflammatory condition” typically refers to a condition characterized by inflammation, or the complex biological response to a noxious stimulus such as damage, auto-immunity, or an infection by a microbial pathogen and/or virus. The clinical features of an inflammatory condition are likely to depend on the noxious stimulus (or stimuli), but is typically characterized by heat, pain, redness and swelling of the affected organ or tissue. The inflammatory condition may be acute or chronic.

In an embodiment, the inflammatory condition is selected from the group consisting of an inflammatory respiratory condition, inflammatory bowel disease and arthritis.

In an embodiment, the inflammatory condition is an inflammatory respiratory condition.

Inflammatory respiratory conditions would be known to persons skilled in the art, illustrative examples of which include ARDS, chronic obstructive pulmonary disease (COPD), asthma, bronchitis, bronchiectasis, sarcoidosis and cystic fibrosis (CF).

In an embodiment, the inflammatory respiratory condition is selected from the group consisting of chronic obstructive pulmonary disease (COPD), asthma, bronchitis, cystic fibrosis (CF) and acute respiratory distress syndrome (ARDS).

In an embodiment, the inflammatory respiratory condition is ARDS. In another embodiment, the inflammatory respiratory condition is sepsis-associated ARDS.

In an embodiment, the inflammatory condition is an inflammatory bowel disease.

The term “inflammatory bowel disease” refers to diseases or disorders that involve chronic inflammation of the digestive tract. Such diseases or disorders would be known to persons skilled in the art, illustrative examples of which include ulcerative colitis, and Crohn's disease.

In an embodiment, the inflammatory condition is arthritis.

The term “arthritis” typically refers to diseases that are characterized by inflammation of the joints that may also include the loss of cartilage. Such diseases would be known to persons skilled in the art, illustrative examples of which include rheumatoid arthritis, osteoarthritis, psoriatic arthritis and ankylosing spondylitis.

In an embodiment, the arthritis is rheumatoid arthritis.

In an embodiment, the inflammatory condition is associated with an increase or upregulation in the level of an inflammatory cytokine selected from the group consisting of IL-1β, IL-6, TNF-α, IL-1α, IL-12(p70), IFN-γ, CXCL-1, MCP-1 and MIP-1α, or combinations thereof.

In an embodiment, the inflammatory condition is associated with an increase or upregulation in the level of an inflammatory cytokine selected from the group consisting of IL-1β, IL-6 and TNF-α, or combinations thereof.

Inflammatory conditions associated with an increase or upregulation in the level of IL-1β, IL-6, TNF-α, IL-1α, IL-12(p70), IFN-γ, CXCL-1, MCP-1 and/or MIP-1α would be known to persons skilled in the art, illustrative examples of which include arthritis (as described by, e.g., Feldman et al., 1996, Annual Review of Immunology, 14: 397-440; McInnes et al., 2007, Nature Reviews Immunology, 7: 429-442; Tanaka et al., 2014, Cold Spring Harbor Perspectives in Biology, 6: a016292-a016295; Woo, 2002, Current Rheumatology Reports, 4: 452-457; Kapoor et al., 2011, Nature Reviews Rheumatology, 7: 33-42), inflammatory bowel disease (as described by, e.g., Neurath, 2014, Nature Reviews Immunology, 14: 329-342; Papadakis and Targan, 2000, The Annual Review of Medicine, 51: 289-298), pain (as described by, e.g., Zhang, 2007, International Anesthesiology Clinics, 45: 27-37), gout (as described by, e.g., Busso, 2010, Arthritis Research & Therapy, 12: 206), fibromyalgia (as described by, e.g., Rodriguez-Pintó et al., 2014, Immunology Letters, 161: 200-203), endometriosis (as described by, e.g., Wu and Ho, 2003, American Journal of Reproductive Immunology, 49: 285-296), chronic obstructive pulmonary disease (as described by, e.g., Chung, 2001, European Respiratory Journal, 18: 50s-59s), asthma (as described by, e.g., Rincon and Irvin, 2012, International Journal of Biological Sciences, 8: 1281-1290; Thomas, 2001, Immunology & Cell Biology, 79: 132-140), alcoholic liver disease (as described by, e.g., McClain et al., 1999, in Seminars in Liver Diseases, 205-220; Kawaratani et al., 2013, Mediators of Inflammation, 2013: 495156), psoriasis/dermatitis (as described by, e.g., Jensen, 2010, Current Opinions in Investigative Drug Discovery, 11: 1211-1220; Baliwag et al., 2015, Cytokine, 31: 781-789; Toshitani et al., 1993, Journal of Investigative Dermatology, 100: 299-304), and lupus (as described by, e.g., Davis et al., 2011, Journal of Interferon & Cytokine Research, 31: 781-789).

### Acute Respiratory Distress Syndrome (ARDS)

The terms “acute respiratory distress syndrome”, “adult respiratory distress syndrome”, or “ARDS” may be used interchangeably herein to refer to an inflammatory condition typically characterized by the disruption of the alveolar-capillary barrier, flooding of protein-rich edema fluid into the alveolar space, and cell recruitment due to immune system stimulation. ARDS can develop after direct lung injuries, e.g., pneumonia, aspiration, inhalation injury, near drowning, pulmonary contusion, reperfusion pulmonary edema and fat embolism. ARDS can also occur during the course of indirect lung injuries, e.g., sepsis, severe trauma, acute pancreatitis, cardiopulmonary bypass, massive transfusions and drug overdose.

“Sepsis-associated acute respiratory distress syndrome” or “sepsis-associated ARDS” typically refers to ARDS that is induced after lung infection or infection at extra-pulmonary sites. An aberrant host response to infection leads to disruption of the pulmonary alveolar-capillary barrier, resulting in lung injury characterized by hypoxemia, inflammation, and non-cardiogenic pulmonary edema.

In an embodiment, the ARDS is sepsis-associated ARDS.

The types of pathogens that can cause sepsis-associated ARDS would be known to persons skilled in the art, illustrative examples of which include bacteria, e.g., Streptococcus pneumonia, Staphylococcus aureus, Pseudomonas aeruginosa, Acinetobacter baumannii; fungi, e.g., Pneumocystis jirovecii, Candida albicans, Candida tropicalis, Candida glabrata; and virus, e.g., cytomegalovirus, influenza, herpes simplex virus-1, respiratory syncytial virus, parainfluenza, human metapneumovirus, enterovirus and coronavirus.

In an embodiment, the sepsis-associated ARDS is caused by a bacterial, fungal or viral infection. In an embodiment, the bacterial infection is selected from the group consisting of Streptococcus pneumoniae, Staphylococcus aureus, Pseudomonas aeruginosa and Acinetobacter baumannii infection. In an embodiment, the viral infection is selected from the group consisting of cytomegalovirus, influenza, herpes simplex virus-1, respiratory syncytial virus, parainfluenza, human metapneumovirus, enterovirus and coronavirus infection. In an embodiment, the fungal infection is selected from the group consisting of Pneumocystis jirovecii, Candida albicans, Candida tropicalis and Candida glabrata infection.

ARDS may be classified using the Berlin Criteria as described by, for example, The ARDS Definition Task Force (2012, American Medical Association, 307(23): 2526-2234), which is based on the ration of arterial oxygen tension and fraction of inspired oxygen when measured at a minimum level of positive end-expiratory pressure (PEEP) of 5 cm H₂O. According to the Berlin Criteria, the severity of ARDS may be classified as mild, moderate or severe (Table 2).

The classification of ARDS based on the Berlin Criteria may also be made in consideration of risk factors and period of exposure to risk factors (i.e., maximum period between exposure to risk factor and ARDS development is 7 days). Risk factors of ARDS include direct risk factors, i.e., pneumonia, aspiration of gastric contents, inhalation injury, pulmonary contusion, lung vasculitis and drowning, and indirect risk factors, i.e., non-pulmonary sepsis, multiple trauma, pancreatitis, non-cardiogenic shock, drug overdose and transfusion-associated acute lung injury (TRALI).

Imaging the lung pathobiology in ARDS patients and measurement of inflammatory markers or other biomarkers for ARDS may also be used in the classification of ARDS.

Suitable imaging methods would be known to persons skilled in the art, illustrative examples of which include computed tomography (CT), as described by, for example, Puybasset et al. (1998, American Journal of Respiratory and Critical Care Medicine, 158(5): 1644-1655). Similarly, suitable inflammatory markers and other biomarkers for ARDS would be known to persons skilled in the art, illustrative examples of which include the biomarkers reviewed by, for example, Blondonnet et al. (2016, Disease Markers, 2016: 35101373).

The present inventors have surprisingly found that CBD and hydroxychloroquine act synergistically to inhibit the production of inflammatory cytokines in response to a noxious stimulus. The present inventors have also surprisingly found that administration of CBD and hydroxychloroquine can treat conditions associated with pulmonary inflammation, including COPD, asthma, CF and ARDS (e.g., by reducing or alleviating symptoms or severity of ARDS, in particular, by reducing the acute pulmonary inflammatory response, reversing edema, and limiting damage to the lung).

### Methods for the Treatment or Prevention of an Inflammatory Condition

In accordance with an aspect disclosed herein, there is provided a method for the treatment of an inflammatory condition comprising administering the composition as described herein to a subject in need thereof.

In another aspect of the present disclosure, there is also provided a use of the composition as disclosed herein in the manufacture of a medicament for the treatment of an inflammatory condition.

The terms “treat”, “treating”, “treatment” and the like are used interchangeably herein to mean relieving, reducing, alleviating, ameliorating or otherwise inhibiting the severity of one or more symptoms of an inflammatory condition in a subject. It is to be understood that the terms “treat”, “treating”, “treatment” and the like, as used herein, do not imply that a subject is treated until the inflammatory condition has been eliminated or are no longer evident. Said treatment may also reduce the severity of the one or more symptoms of an inflammatory condition.

The terms “prevent”, “preventing”, “prevention” and the like are used interchangeably herein to mean preventing the establishment of a condition of a disease, or to otherwise prevent, hinder, retard, abrogate or reverse the onset or progression of a condition or disease or other undesirable symptoms in any way whatsoever.

The term “subject” as used herein refers to any mammal, including livestock and other farm animals (such as cattle, goats, sheep, horses, pigs and chickens), performance animals (such as racehorses), companion animals (such as cats and dogs), laboratory test animals and humans. In an embodiment, the subject is a human.

Changes in the symptoms or severity of an inflammatory condition as measured by any quantitative methods or clinical instruments known in the art (e.g., reduction in inflammatory mediators, reduction in white blood cell count and/or neutrophil infiltration, reduction in acute pulmonary inflammatory response, reversal of edema, and limiting damage to the lung). Said beneficial or desired therapeutic outcomes may be quantified by measuring clinical parameters, illustrative examples of which include the measurement of oxygenation index (OI[F_{IO2}×mean airway pressure×100)/Pa_{O2}]) or oxygenation saturation index (OSI[F_{IO2}×mean airway pressure×100)/oxygen saturation by pulse oximetry (Sp_{O2}] as described by Des Prez et al. (2017, Chest, 152(6): 1151-1158), determination of the ratio of arterial oxygen tension and fraction of inspired oxygen (PaO₂:FiO₂) as described by The ARDS Definition Task Force (2012, supra), detection of bilateral pulmonary opacities using chest radiography, detection of altered levels of immunological biomarkers as described by Blondonnet et al. (2016, supra), endoscopic and histological evaluation of colonic mucosa and stool analysis (including fecal occult blood score and stool consistency). Subjective measures of said beneficial or desired therapeutic outcomes can also be made using clinical instruments known in the art, illustrative examples of this include the Lung Injury Score (LIS) (Murray et al., 1988, American Review of Respiratory Disease, 138(3): 720) the American-European Consensus Conference (AECC) Definition (Bernard et al., 1994, American Journal of Respiratory and Critical Care Medicine, 149: 818-824), illness severity scores (e.g., Acute Physiology and Chronic Health Evaluation II (APACHE II) and Simplified Index Score II), the Berlin Criteria (The ARDS Definition Task Force, 2012, supra), Crohn's Disease Activity Index (CDAI; Best et al., 1976, Gastroenterology, 70: 439-444) and Mayo Score for Ulcerative Colitis (UC; Lewis et al., 2008, Inflammatory Bowel Disease, 14: 1660-1666)

In an embodiment, the methods disclosed herein reduce in the symptoms or severity of an inflammatory condition by at least 10%, preferably at least 20%, preferably at least 30%, preferably at least 40%, preferably at least 50%, preferably at least 60%, preferably at least 70%, preferably at least 80%, preferably at least 90%, or more preferably at least 100% as compared to a subject with the same inflammatory condition who has not been administered the composition.

In an embodiment, the composition is for use in the treatment or prevention of an inflammatory condition.

In an embodiment, the composition is for use in the treatment or prevention of an inflammatory respiratory condition.

In an embodiment, the composition is for use in the treatment or prevention of an inflammatory respiratory condition selected from the group consisting of ARDS, COPD, asthma, bronchitis, and CF.

In an embodiment, the composition is for use in the treatment or prevention of ARDS.

In an embodiment, the composition is for use in the treatment or prevention of sepsis-associated ARDS.

In an embodiment, the composition is for use in the treatment or prevention of sepsis-associated ARDS caused by a bacterial, fungal, or viral infection, as described elsewhere herein.

In an embodiment, the composition is for use in the treatment or prevention of inflammatory bowel disease.

In an embodiment, the composition is for use in the treatment or prevention of arthritis. In another embodiment, the composition is for use in the treatment or prevention of rheumatoid arthritis.

In an aspect disclosed herein, there is provided a use of the composition described herein in the manufacture of a medicament for the treatment or prevention of an inflammatory condition.

In an embodiment, the inflammatory condition is an inflammatory respiratory condition.

In an embodiment, the inflammatory respiratory condition is selected from the group consisting of ARDS, COPD, asthma, bronchitis and CF.

In an embodiment, the inflammatory condition is ARDS. In another embodiment, the ARDS is sepsis-associated ARDS.

In another embodiment, the inflammatory condition is inflammatory bowel disease.

In another embodiment, the inflammatory condition is arthritis. In another embodiment, the arthritis is rheumatoid arthritis.

In some embodiments, periodic re-administration of the composition may be required to achieve a desirable therapeutic effect. The exact amounts and rates of administration of the composition will depend on a number of factors, examples of which are described elsewhere herein, such as the subject's age, body weight, general health, sex and dietary requirements, as well as any drugs or agents used in combination or coincidental with the administration of the composition. Where multiple doses are required, these may be administered hourly, daily, weekly, monthly or at other suitable time intervals or the dose may be proportionally reduced as indicated by the exigencies of the situation.

In an embodiment, the composition is administered twice per day.

In an embodiment, the composition is administered for a period of at least one week.

In an embodiment, the composition is administered to the subject for a period of between 1 to 10 weeks (e.g., for 1 week, 2 weeks, 3 weeks, 4 weeks, 5 weeks, 6 weeks, 7 weeks, 8 weeks, 9 weeks, or 10 weeks).

Thus, in an embodiment, the composition is administered to the subject for a period of between 1 to 10 weeks, preferably for about 1 week, preferably about 2 weeks, preferably about 3 weeks, preferably about 4 weeks, preferably about 5 weeks, preferably about 6 weeks, preferably about 7 weeks, preferably about 8 weeks, preferably about 9 weeks, or more preferably for about 10 weeks.

In an embodiment, the composition is administered to the subject for a period of between 3 to 4 weeks.

### Methods for Modulating an Immune Response

In another aspect disclosed herein, there is provided a method of modulating an immune response comprising administering to a subject in need thereof the composition as described herein.

Persons of ordinary skill in the art would appreciate that modulation of the immune response in accordance with the methods disclosed herein can be determined by a variety of methods known in the art, illustrative examples of which include measuring changes in cytokine production (e.g., levels, concentrations, ratios) in the subject such as before and after treatment or during the course of treatment. The term “modulation”, as used herein, is to be understood to mean a reduction or an increase in the immune response, as determined, for example, by a decrease or increase in the level, concentration and/or ratio of inflammatory mediators, such as cytokines.

The terms “level” and “amount” are used interchangeably herein to refer to a quantitative amount (e.g., moles or number), a semi-quantitative amount, a relative amount (e.g., weight %, or mole % within a class, or a ratio), a concentration, and the like. Thus, these terms encompass absolute or relative amounts or concentrations, including of inflammatory mediators, in a sample.

Inflammatory mediators, such as cytokines, may be quantified or detected using any suitable technique, including, but not limited to, nucleic acid- and protein-based assays. In illustrative nucleic acid-based assays, nucleic acid is isolated from cells contained in a biological sample according to standard methodologies (Sambrook, et al., 1989, Molecular Cloning: A Laboratory Manual; and Ausubel et al., 1994, Current Protocols in Molecular Biology).

In other illustrative embodiments, protein levels of inflammatory mediators can be measured using protein-based assays known in the art. For example, an antibody-based technique may be employed to determine the level of an autoantibody in a sample, illustrative examples of which include immunoassays, such as the enzyme-linked immunosorbent assay (ELISA), immunohistochemistry (IHC) and the radioimmunoassay (RIA).

In an embodiment, protein expression is measured using a multiplexed protein expression analysis method. In another embodiment, the multiplexed protein expression analysis method is a protein microarray or Luminex bead array.

Protein-capture arrays that permit simultaneous detection and/or quantification of a large number of proteins may also be employed. For example, low-density protein arrays on filter membranes, such as the universal protein array system allow imaging of arrayed antigens using standard ELISA techniques and a scanning charge-coupled device (CCD) detector. Exemplary protein capture arrays include protein function arrays comprising spatially addressed protein-binding molecules (i.e., antigens), which can facilitate extensive parallel analysis of autoantibodies with specificity for the antigens that comprise the protein function array. Central to this type of analysis is the retention of the correctly folded protein confirmation of the arrayed antigen. Protein function arrays have been shown to have the required properties of specificity and acceptable background, and are available commercially (e.g., Sengenics). Various methods for the preparation of protein function arrays have been reported (see, e.g., Gnjatic et al., 2009, Journal of Immunological Methods, 341(50): 1-2; PCT/GB01/00395, PCT/GB02/05499, PCT/GB03/00362). Individual spatially distinct functional proteins are typically attached to a support surface, which is generally planar or contoured. Common physical supports include glass slides, silicon, microwells, nitrocellulose or PVDF membranes, and magnetic and other microbeads.

Particles in suspension can also be used as the basis of arrays, providing they are coded for identification; systems include colour coding for microbeads (e.g., available from Luminex, Bio-Rad and Nanomics Biosystems) and semiconductor nanocrystals (e.g., QDots™, available from Quantum Dots), and barcoding for beads (UltraPlex™, available from Smartbeads) and multimetal microrods (Nanobarcodes™ particles, available from Surromed). Beads can also be assembled into planar arrays on semiconductor chips (e.g., available from LEAPS technology and BioArray Solutions). Where particles are used, individual protein-capture agents (e.g., antibodies to inflammatory mediators or inflammatory mediator-binding fragments thereof) are typically attached to an individual particle to provide the spatial definition or separation of the array. The particles may then be assayed separately, but in parallel, in a compartmentalised way, for example in the wells of a microtiter plate or in separate test tubes.

In an illustrative example, a patient or control sample is delivered to a protein function array under conditions suitable for protein or peptide binding, and the array is washed to remove unbound or non-specifically bound components of the sample from the array. Next, the array is incubated with fluorescently-labelled antibody to detect the interaction between array antigens and inflammatory mediator(s) present in the sample. The presence or amount of protein or peptide bound to each feature of the array is detected using a suitable fluorescence detection system. The amount of protein bound to a feature of the array is proportional to the intensity of fluorescence. In certain embodiments, local background fluorescence obtained from control features of the array are automatically subtracted and relative fluorescent units (rfu) for each feature of the array is recorded.

In some embodiments, the protein function array is a Luminex-based multiplex assay, which is a bead-based multiplexing assay, where beads are internally dyed with fluorescent dyes to produce a specific spectral address. Biomolecules (such as an oligo or antibody) can be conjugated to the surface of beads to capture analytes of interest; that is, inflammatory markers, e.g., cytokines. Flow cytometric or other suitable imaging technologies known to persons skilled in the art can then be used for characterisation of the beads, as well as for detection of analyte presence. The Luminex technology enables are large number of proteins, genes or other gene expression products (e.g., 100 or more, 200 or more, 300 or more, 400 or more) to be detected using very small sample volume (e.g., in a 96 or 384-well plate).

In some embodiments, the level of an inflammatory mediator can be normalised against a housekeeping biomarker. The term “housekeeping biomarker” refers to a biomarker or group of biomarkers (e.g., polynucleotides and/or polypeptides), which are typically found at a constant level in the cell type(s) or tissue(s) being analysed and across the conditions being assessed.

In other embodiments, the level of an inflammatory mediator measured using a protein array can be normalised by both intra- and inter-array data normalisation. For example, the overall median value of all median relative fluorescent units (rfu) of each protein in a protein function array (excluding data from control proteins) is calculated and intra-array normalisation achieved by dividing the median of the quadruplicate spots of each protein on the array, by the overall median value of all the proteins on the array in each sample. Inter-array normalisation can be achieved using bioinformatics software packages that are known in the art. For example, inter-array normalisation can be achieved using the normalize.quantiles package in R (Bolstad et al., 2003, Bioinformatics, 19(2): 185-193).

It would be understood by those skilled in the art, as described elsewhere herein, that the method of analysing the level of an inflammatory mediator in a sample can be quantitative, semi-quantitative or qualitative in nature. For example, quantitative analyses will typically provide a concentration or number of an inflammatory mediator nucleic acid molecule or protein in the sample within an appropriate error margin (e.g., mean+/−standard deviation). By contrast, semi-quantitative or qualitative analyses will typically provide an indication of the relative amount of an inflammatory mediator in a sample. This may involve a comparison of an amount of an inflammatory mediator in a first sample with an amount of an inflammatory mediator in a second sample and making a determination as to the relative amount of the inflammatory mediator between the first and second samples.

It will be understood by persons skilled in the art that, where a comparison is made to a reference value, then the manner in which the sample is assessed for the level of the one or more inflammatory mediators should be substantially identical to the manner in which the reference value is derived in order to ensure that an appropriate comparison can be made for the purposes of determining whether or not the immune response has been modulated in a subject following administration of CBD and hydroxychloroquine

In an embodiment, the method of modulating an immune response comprises decreasing the level of an inflammatory mediator relative to a reference level.

In an embodiment, the inflammatory mediator is a cytokine.

The term “cytokine” as used herein refer to factors that exert a variety of effects on cells, for example, inducing growth or proliferation, illustrative examples of which include IL-1β, IL-2, IL-10, IL-12 (p70), IFN-γ, TNF-α, IL-1α, IL-3, IL-4, IL-5, IL-6, IL-9, IL-12(p40), IL-13, IL-17A, Eotaxin, G-CSF, GM-CSF, IFN-γ, KC, MCP-1(MCAF), MIP-1α, MIP-1β, RANTES and CXCL-1.

In an embodiment, the method of modulating an immune response comprises decreasing the level of an inflammatory cytokine selected from the group consisting of IL-1β, IL-6, TNF-α, IL-1α, IL-12(p70), IFN-γ, CXCL-1, MCP-1 and MIP-1α, relative to a reference level.

In an embodiment, the method of modulating an immune response comprises decreasing the level of an inflammatory cytokine selected from the group consisting of IL-1β, IL-6 and TNF-α relative to a reference level.

Those skilled in the art will appreciate that the invention described herein is susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Unless otherwise defined, all technical and scientific terms used herein have the same meanings as commonly understood by one of ordinary skill in the art to which this invention belongs.

All patents, patent applications and publications mentioned herein are hereby incorporated by reference in their entireties.

The various embodiments enabled herein are further described by the following non-limiting examples.

### EXAMPLES

### Materials

### Active Agents

Cannabidiol (CBD) and hydroxychloroquine (HCQ) were purchased from a commercial supplier and provided to Pharmacology Discovery Services Taiwan, Ltd. The formulations are summarised in Table 3.

### Chemicals

0.9% NaCl (Sintong Chemical Industry Co., Ltd. Taiwan), Bio-Plex mouse cytokine Th1-7plex panel (Bio-Rad, USA), Bio-Plex mouse cytokine group I 23-plex panel (Bio-Rad, USA), Lipopolysaccharide (Escherichia coli 055:B5, Sigma L-2880, USA), Mouse D-Dimer (D2D) ELISA kit (MyBioSource, USA), Phosphate buffered saline (Sigma, USA) and Water for injection (WFI) (Tai-Yu, Taiwan).

### Methods

### Lipopolysaccharide (LPS)-Induced Sepsis

Male C57BL/6 mice weighing 10-20 g were used. Vehicle and active agents (i.e., CBD and hydroxychloroquine) alone or in combination were administered intraperitoneally (IP) or orally at 1 hour before LPS (100 μg/100 μL/mouse) injected intravenously (IV). At 2 hours after LPS injection, blood was collected from all mice by cardiac puncture, sera processed and assayed for biomarkers using either the Th17-plex for IL-1β, IL-2, IL-10, IL-12 (p70), IFN-γ and TNF-α by Luminex, plus IL-6 and D-dimer by ELISA, or the 23-plex for IL-1α, IL-1β, IL-2, IL-3, IL-4, IL-5, IL-6, IL-9, IL-10, IL-12(p40), IL-12(p70), IL-13, IL-17A, Eotaxin, G-CSF, GM-CSF, IFN-γ, KC, MCP-1(MCAF), MIP-1α, MIP-10, RANTES, and TNF-α by Luminex, plus IL-6 by ELISA. ANOVA followed by Dunnett's test was applied for comparison between vehicle and treatment groups. p<0.05 is considered significant.

For synergy analysis, data were baseline subtracted using the cytokine levels of sham treated mice (i.e., no LPS injection), before normalizing the values for each cytokine relative to maximum values across the groups. The normalized values were used to calculate the relative inhibition where a value of 1 represents complete inhibition and a value of 0 represents no inhibition. Synergy was calculated using the statistical methods described elsewhere herein.

### LPS-Induced Pulmonary Inflammation

Male C57BL/B6 mice weighing 10-20 g were used. Vehicle and active agents (i.e., CBD and hydroxychloroquine) alone or in combination were administered intraperitoneally (IP) at 1 hour before LPS (˜80 mg/kg) was administered intratracheally. 24 hours after LPS injection, mice were anaesthetized with pentoparbital and 0.5 mL of PBS was administered twice through a tracheal cannula after which approximately 0.6 mL of broncheoalveolar lavage fluid (BALF) was obtained. The BALF was assayed for IL-10, TNF-α, MCP-1(MCAF) and CXCL-1 by Luminex. ANOVA followed by Dunnett's test was applied for comparison between vehicle and treatment groups. p<0.05 was considered significant.

In a separate test group of animals with pulmonary inflammation induced as detailed above, lungs were harvested at termination (five lobes per lung) and fixed in 10% neutral buffered formalin for histopathology. Samples were sectioned at 4-6 μm and stained with hematoxylin and eosin (H&E) to examine inflammatory lesions as described by Shackleford et al. (2002, Toxicologic Pathology, 30(1): 93-96). Briefly, a score of 0 corresponds to not present, and a score of 5 indicates a severe/high number of lesions. Scores were averaged across lobes for each animal and then across animals for each treatment group.

For synergy analysis, data were baseline subtracted using the cytokine levels of sham treated mice (i.e., no LPS injection), before normalizing the values for each cytokine relative to maximum values across the groups. The normalized values were used to calculate the relative inhibition where a value of 1 represents complete inhibition and a value of 0 represents no inhibition. Synergy was calculated using the statistical methods described elsewhere herein.

### 2,4,6-Trinitrobenzene Sulfonic Acid (TNBS)-Induced Colitis

Male BALB/c were used as an in vivo model of colitis, induced by intracolonic administration of 2,4,6-trinitrobenzene sulfonic acid (TNBS) in 50% ethanol, as previously described by Antoniou et al. (2016, Annals of Medicine and Surgery, 11: 9-15). Briefly, the ethanol disrupts the intestinal barrier, permitting TNBS to interact with colon proteins. Interaction of TNBS with high-molecular weight proteins renders them immunogenic, leading to Th1 mediated inflammation, which is causative of symptoms include inconsistent stool formation and blood in the faeces.

Vehicle and active agents (i.e., CBD and hydroxychloroquine) alone or in combination were administered intraperitoneally (IP) once a day starting from Day 1 (i.e., 24 hours before TNBS) to Day 4, for a total of 4 consecutive days (Days 1-4). On the Day of TNBS challenge (i.e., Day 2), test articles and vehicle were given 2 hrs before TNBS.

Colon tissues were harvested in all animals at termination. Colon samples were taken at 0.5, 2, and 3.5 cm from the anus, fixed in formalin, and then embedded in paraffin blocks. Four-micrometer tissue sections were cut and stained with H&E for histological analysis in accordance with the method of Dieleman et al. (1998, Clinical Experimental Immunology, 114: 385-391). Histological criteria included: abnormalities of mucosal architecture, extent of inflammation, erosion or ulceration, epithelial regeneration, and the percentage involvement by the disease process. The scoring was based on the findings of the observers by examining three sections from each colon per animal. Total score for colitis (Total Colitis Index) were added, resulting in a combined histological score range from 0 to 60.

Other endpoint measures of stool consistency score (i.e., 0—normal stools, 1, soft but still formed stools, 2—very soft stools, 3—diarrhoea), faecal occult blood score (i.e., 0—negative hemoccult, 1—positive hemoccult, 2—blood traces in stool visible, 3—rectal bleeding), colon weight, colon macroscopic damage score (a composite of adhesions, strictures, ulcers/inflammations and wall thickness) and myeloperoxidase levels in the colon tissue were also assessed. The data from each of these endpoints was baseline subtracted using the equivalent measures from sham treated mice (i.e., no TNBS) and reduction in any measure calculated as a relative reduction. Synergy was calculated using the statistical methods described elsewhere herein.

### Collagen-Induced Arthritis

Female Lewis rats were challenged with porcine type-II collagen with Freund's adjuvant on Day 1 (0.2 mg/0.2 mL/rat) by subcutaneous injection at the base of the tail to induce arthritis. A booster injection at 0.1 mg/0.1 mL/rat) was administered on Day 7. On Day 16, rats were allocated into groups of six based on hind paw volume, with animals allocated into groups to provide a similar distribution of hind paw volumes.

Vehicle and active agents (i.e., CBD and hydroxychloroquine) alone or in combination were administered intraperitoneally (IP) once a day starting from Day 17 to Day 30, i.e., for a total of 14 consecutive days.

Disease was assessed by measuring hind paw volume with plethysmometer and using a qualitative severity score system as outlined in Table 16 on Day 1, 7, 10, 14, 16, 18, 20, 22, 24, 26, 28 and 30.

Post-termination on Day 30, blood was collected from all rats and analysed for levels of the inflammatory cytokines IL-10, IL-6 and TNF-α by ELISA. Both hind paws were harvested, weighed and formalin-fixed for histopathology. Tissue was assessed to evaluate cartilage and bone destruction by pannus formation and mononuclear cell infiltration in synovial tissues according to the scoring matrices set out in Tables 17 and 18. A total histology score, being the sum of the pannus formation and mononuclear cell infiltration score, was also calculated.

For synergy analysis, data were baseline subtracted using sham treated mice (i.e., no collagen injection), before normalizing the values relative to vehicle control. Synergy was calculated using the statistical methods described elsewhere herein.

### In Vitro Analysis of the Anti-Inflammatory Activity of the Combination of CBD and Hydroxychloroquine

The anti-inflammatory activity the combination of CBD and hydroxychloroquine was assessed by determining cytokine release from human peripheral blood mononuclear cells (PBMCs) stimulated with bacterial lipopolysaccharide (LPS) using a Luminex based assay. A 96-well microtitre plate-based checkerboard assay with seven concentrations (including the no drug control) of CBD and hydroxychloroquine were assessed in combination was used to determine the drug-drug interaction. Briefly, frozen PMBCs from two independent donors were thawed, diluted in culture medium, seeded into 96-well microtitre plates and incubated at 37° C., 5% CO₂ for 1 hour prior to the addition of test compounds. After the addition of the test compounds the cells were returned to the incubator for 1 hour, after which LPS was added to the wells. Plates were incubated at 37° C., 5% CO₂ for 24 hours. After 24 hours, cell culture supernatants were removed and analysed for cytokine levels using Luminex methodology according to manufacturers' instructions. All values had the vehicle background subtracted prior to further analysis. Three plates were set up in parallel using the PBMCs from the same donor. Inhibition of cytokine release was determined relative to the no treatment control (i.e., that was stimulated with LPS) and averaged across the three replicates. A cytotoxicity assay using AlmarBlue with the identical plate set up was performed in parallel.

### Statistical Analysis

Drug synergy was determined using the Bliss Independence method where the predicted effect of the drug combination is calculated using the equation:

E_{pred A+B}(E_{A}+E_{B})−(E_{A}E_{B})

Synergy values for each combination of drug concentrations were calculated using the Excess Over Bliss method (the difference between the observed and predicted inhibition) described by, e.g., Liu et al. (2018, Statistics in Biopharmaceutical Research, 10: 112-122, where a value greater than 0 is indicative of synergy and the higher the value the stronger the synergy.

### Example 1—Combination of CBD and Hydroxychloroquine Synergize to Modulate an Inflammatory Response In Vitro

CBD and hydroxychloroquine act synergistically to inhibit LPS-induced production of the inflammatory cytokines IL-1β, IL-6, TNF-α, IL-1α, and MIP-1α at multiple drug concentrations. PBMCs from Donor 1 were treated with 5 μg/mL CBD in combination with 0.63, 1.25, 2.5, 5 and 10 μg/mL hydroxychloroquine, the results of which are shown in Table 19. PBMCs from Donor 2 were treated with 5 μg/mL CBD in combination with 0.63, 1.25, 2.5, 5, 10 and/or 20 μg/mL hydroxychloroquine, the results of which are shown in Table 4.

Values presented herein are expressed as inhibition relative to the untreated control, i.e. a value of 0 is no inhibition and a value of 1 is complete inhibition of release of the relevant cytokine. Cell viability was greater than 84% in all of the treatments listed in Tables 3 and 4.

### Example 2—Combination of CBD and Hydroxychloroquine Synergize to Modulate an Inflammatory Response In Vivo

CBD and hydroxychloroquine act synergistically to inhibit the LPS-induced production of inflammatory cytokines IL-1β, IL-6, IL-12(p70), IFN-γ, and/or TNF-α at multiple drug concentrations (Tables 5-8).

### Example 3—Combination of CBD and Hydroxychloroquine Synergize to Modulate the Immune Response in an In Vivo Model of Pulmonary Inflammation

Mice were challenged with LPS intratracheally to trigger pulmonary inflammation in a manner recapitulates clinical aspects of pulmonary inflammation observed inflammatory respiratory conditions such as COPD (see, e.g., Hakannsson et al., 2012, Pulmonary Pharmacology & Therapeutics, 25: 399-406).

CBD and hydroxychloroquine act synergistically to inhibit the production of inflammatory cytokines IL-1β, IL-6, TNF-α, CXCL-1 and MCP-1 at multiple concentrations, as measured from the BALF of mice with LPS-induced pulmonary inflammation (Tables 9-10).

BALF collected from mice were also analysed for white blood cell (WBC) count using an automatic haematology analyser. Cell counts were normalised using sham treated mice, and analysed relative to the highest values across the groups (Table 11). These data demonstrate that the combination of CBD and hydroxychloroquine reduced total WBC counts and neutrophil levels to a greater extent than either CBD or hydroxychloroquine alone.

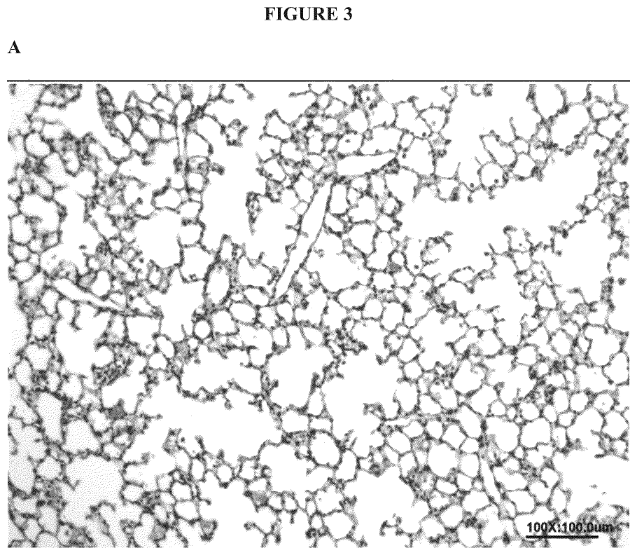

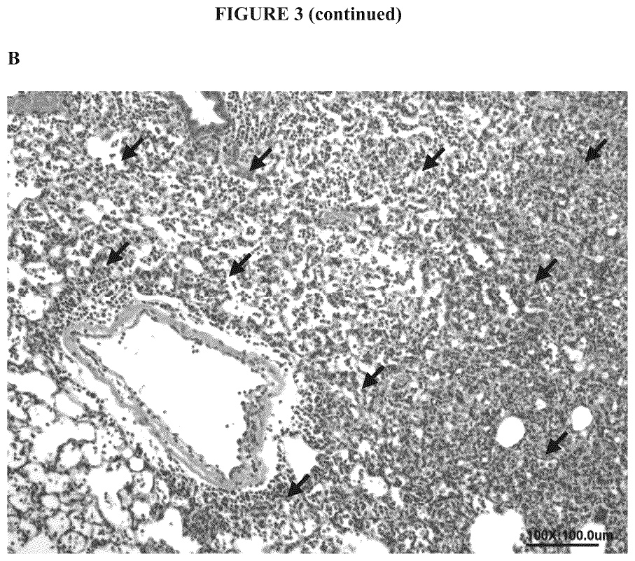

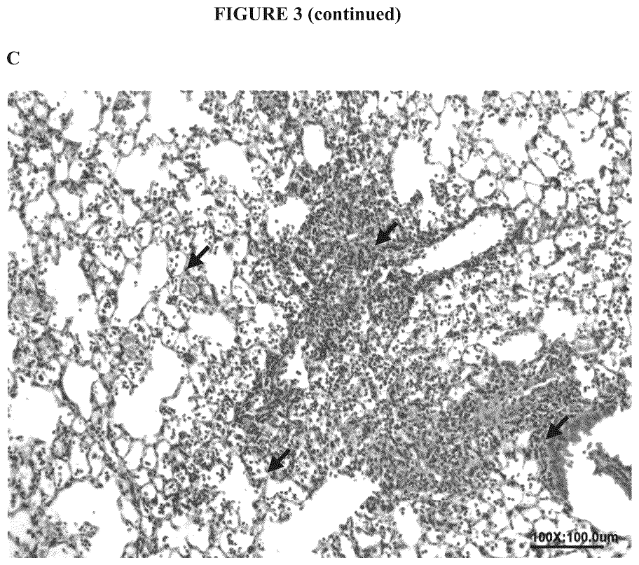

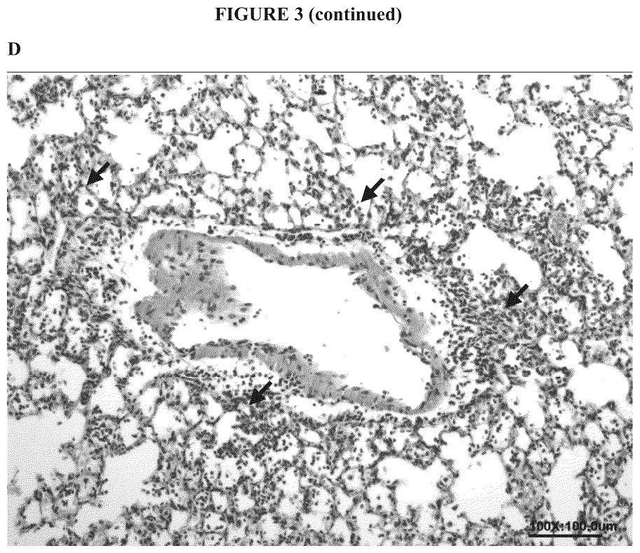

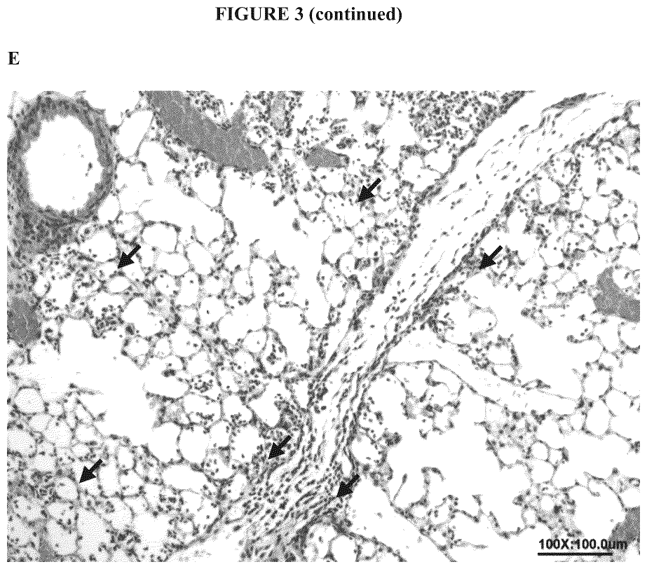

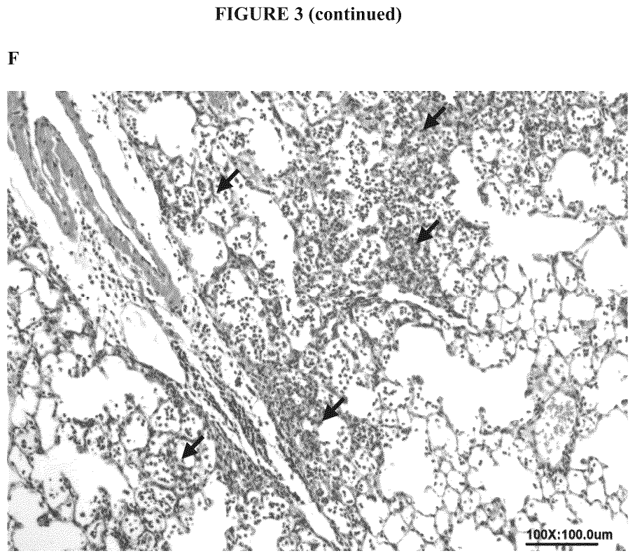

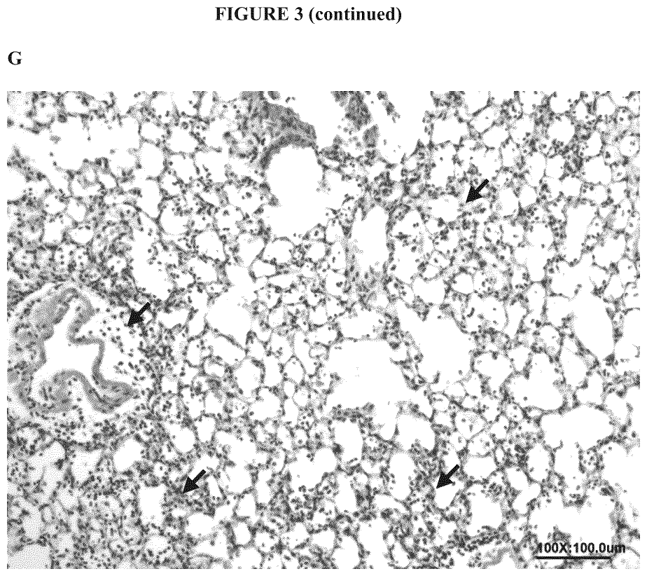

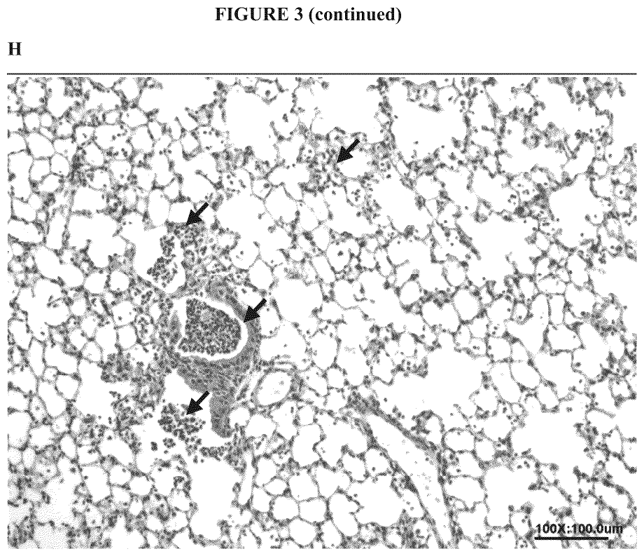

The combination of CBD and hydroxychloroquine also outperformed either CBD or hydroxychloroquine alone at equivalent doses in reducing inflammatory lesions in the lungs of mice with LPS-induced pulmonary inflammation (Tables 14 and 15; FIG. 3).

### Example 4—Combination of CBD and Hydroxychloroquine Synergize to Treat Inflammatory Bowel Disease In Vivo

CBD and hydroxychloroquine act synergistically to reduce the myeloperoxidase (MPO) levels in the colon tissue, stool consistency score and macroscopic damage score (Table 11).

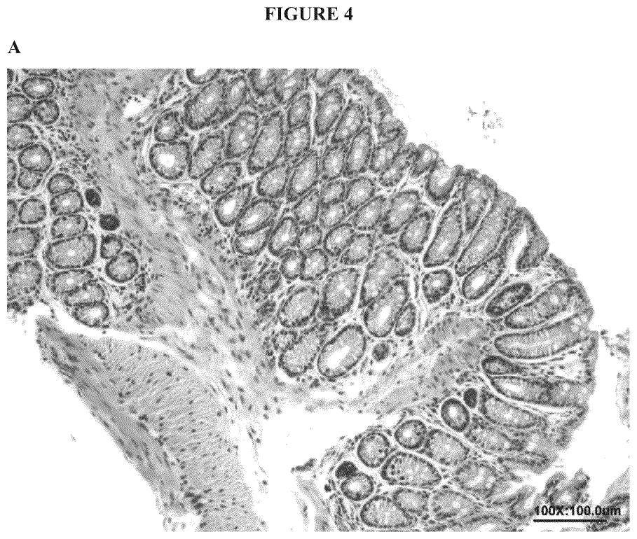

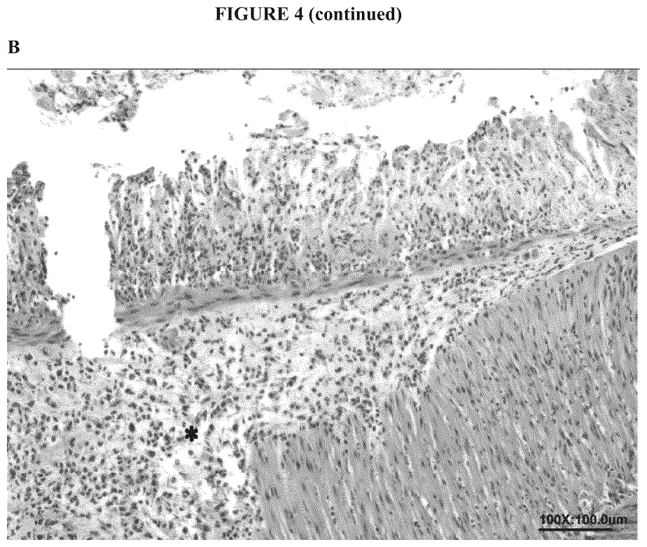

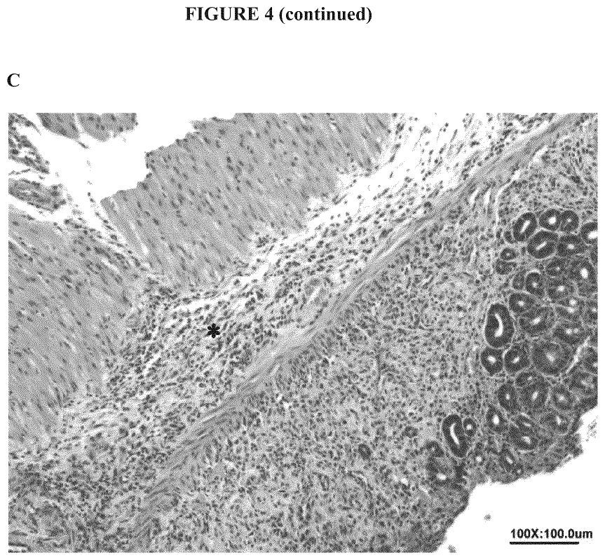

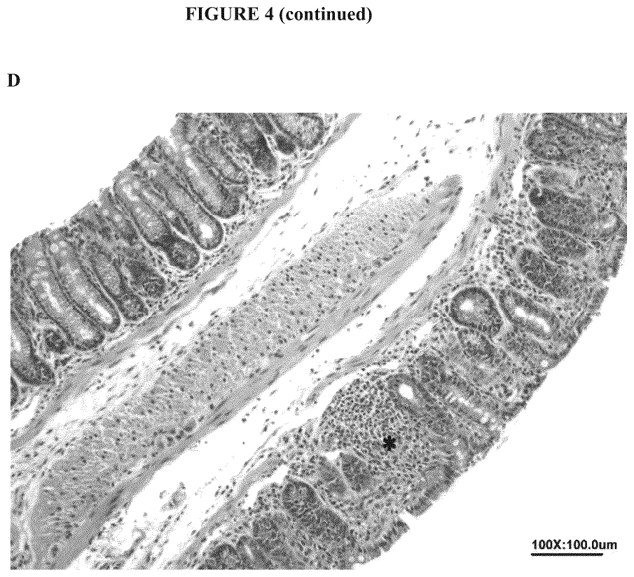

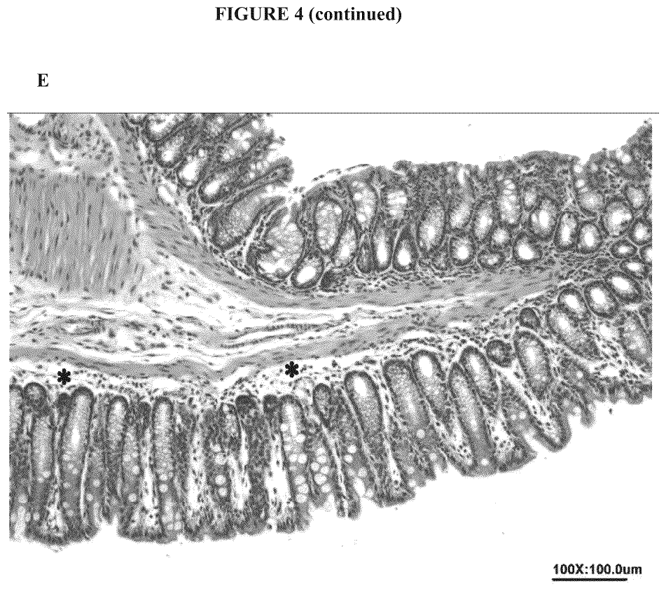

The combination of CBD and hydroxychloroquine was shown to outperform each drug alone in relation to a reduction in Total Colitis Index, as assessed by histological analysis of changes to the colitis of the distant colon of mice with TNBS-induced colitis (Tables 12). Representative stained sections are presented in FIG. 4. The combination of CBD and hydroxychloroquine also improved colon weight to body weight ratio (Table 12), as compared to the administration of either drug as a single agent.

### Example 5—Combination of CBD and Hydroxychloroquine Synergize to Treat Arthritis In Vivo

CBD and hydroxychloroquine at doses of 1 mg/kg and 2.5 mg/kg, respectively, act synergistically to reduce clinical score and paw volume at Day 24, together with pannus formation and total histological score at endpoint (i.e., Day 30). The Excess Over Bliss scores for clinical score, paw volume, pannus formation and total histological score were 0.05, 0.26, 0.30 and 0.03, respectively (Table 19).

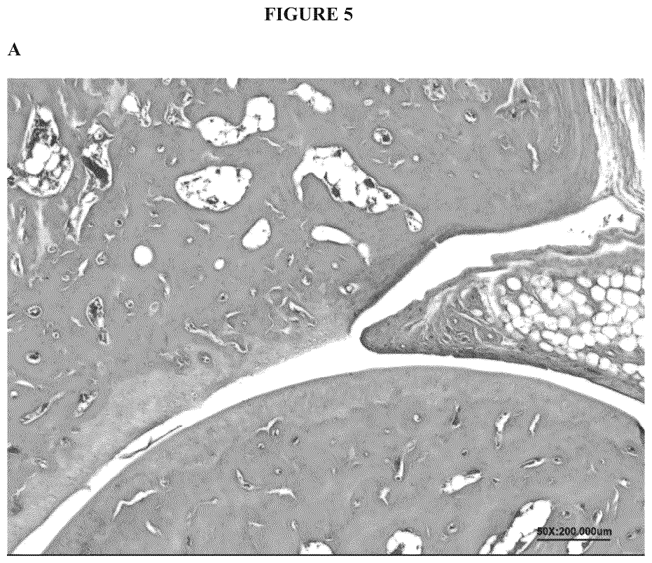

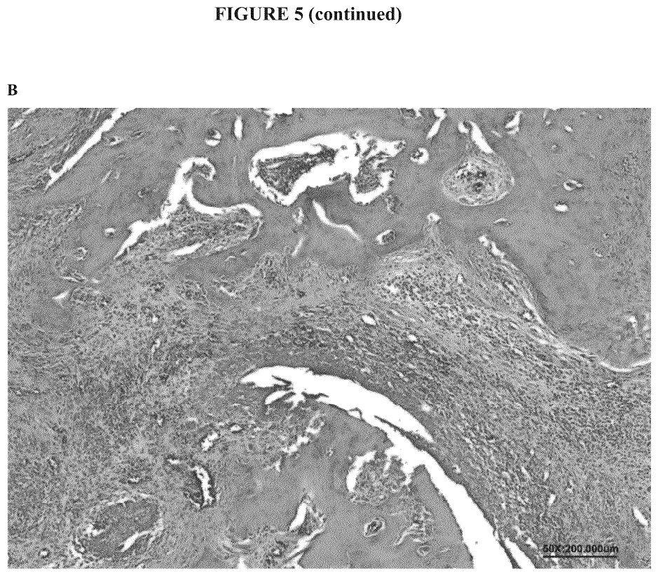

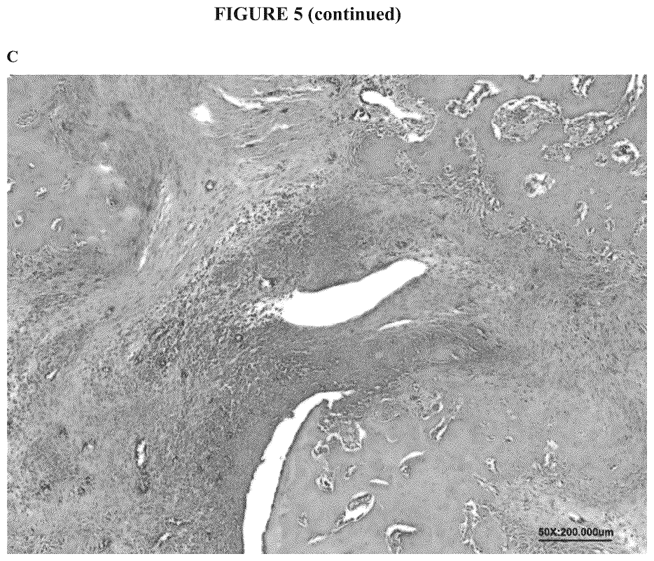

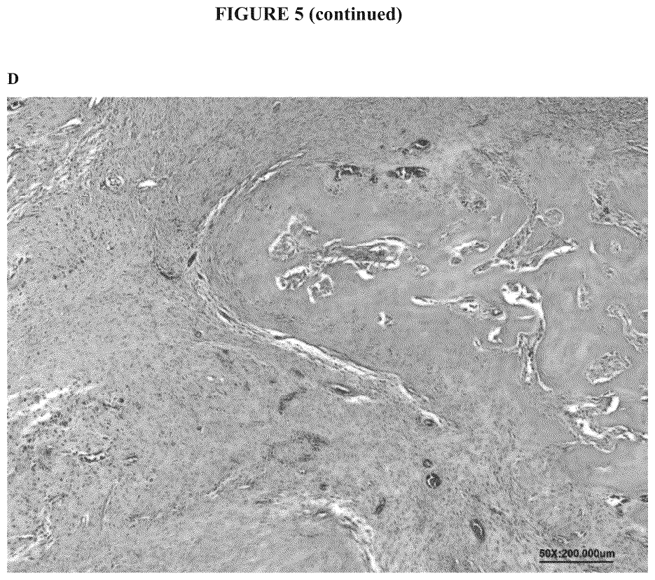

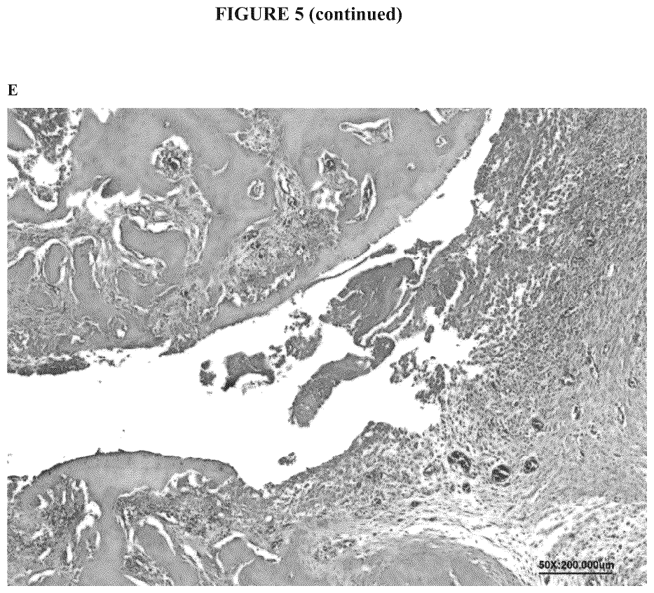

The combination of 1 mg/kg CBD and 2.5 mg/kg hydroxychloroquine was shown to outperform each drug alone in reducing levels of the inflammatory cytokines IL-1β and IL-6 in serum (Table 20; FIG. 5).

Hydroxychloroquine has been used for the treatment of rheumatoid arthritis in the form of hydroxychloroquine sulfate. However, long-term use of hydroxychloroquine has been associated with ocular toxicity and cardiac effects (e.g., cardiomyopathy and QT prolongnation). Clinically, the most important predictor of ocular toxicity and cardiac effects in rheumatoid arthritis patients is the cumulative dose of hydroxychloroquine. To understand the capacity of CBD to permit the reduction of hydroxychloroquine, while retaining therapeutic effect, the results obtained using 1 mg/kg CBD in combination with 2.5 mg/kg hydroxychloroquine (i.e., low dose HCQ) was compared to that 25 mg/kg hydroxychloroquine alone (i.e., high dose HCQ). The combination of 1 mg/kg CBD and low dose HCQ was more, or similarly effective in reducing arthritis across all assessments, with the exception of mononuclear cell infiltration (Table 21). These data indicate that the combination of CBD with hydroxychloroquine allows for a ten-fold reduction in the dose of hydroxychloroquine without sacrificing therapeutic efficacy.

### Example 6—Exemplary Compositions Comprising CBD and Hydroxychloroquine

Exemplary compositions according to the present disclosure comprise the ingredients in Tables 22 and 23.

Both the first and the second capsule according to this example are soft gelatin capsules. However, other exemplary embodiments described herein comprise a first capsule being a soft gelatin capsule and a second capsule being a polymer capsule.

The solid dosage form of hydroxychloroquine according to this example is a solid tablet comprising a number of inactive ingredients (i.e., excipients) including the following:
Calcium Phosphate, Dibasic, Anhydrous (UNII: L11K75P92J);
HYPROMELLOSE 2910 (6 MPA.S) (UNII: 0WZ8WG20P6);
Magnesium Stearate (UNII: 70097M6I30);
Polyethylene glycol 400 (UNII: B697894SGQ);
Polysorbate 80 (UNJI: 60ZP39ZG8H);
Starch, Corn (UNII: 08232NY3SJ);
Titanium Dioxide (UNII: 15FIX9V2JP);
CARNAUBA WAX (UNII: R12CBM0EIZ); and
FERROSOFERRIC OXIDE (UNII: XM0M87F357).

As described elsewhere herein, the compositions may be produced according to the methods disclosed in U.S. Pat. Nos. 9,433,584, 10,383,826 and WO 2012/017325. As an exemplary composition described herein comprises a tablet solid dosage form (see, e.g., FIG. 1) of hydroxychloroquine, with capsules are of the same material, such exemplary compositions may be produced according to the methods described in U.S. Pat. Nos. 9,433,584 and 10,383,826 as they relate to a “double” soft gel capsule (see, e.g., U.S. Pat. No. 9,433,584, column 6, lines 25-50).

### Example 7—Manufacture of Fixed Dose Combination Product

An exemplary fixed dose combination product (i.e., composition) comprising a film coated hydroxychloroquine tablet contained within a soft gel capsule comprising CBD were manufactured under Good Manufacturing Practice (GMP) certified conditions by ProCaps (Barranquilla, Colombia). The manufacturing process consists of three steps: (i) manufacture of the solid dosage form of hydroxychloroquine (e.g., tablet); (ii) manufacture of the CBD oil solution; and (iii) combination of the solid dosage form of hydroxychloroquine and CBD oil solution within a soft gel capsule.

### Hydroxychloroquine Sulfate Tablet

Each hydroxychloroquine tablet contained 100 mg of active pharmaceutical ingredient (API) and the inactive ingredients as listed in Table 24. The hydroxychloroquine, calcium phosphate, pre-gelatinized starch and 50% of the croscarmellose sodium were first blended as dry powders. Water was then added to 10-15% to act as a binding agent while mixing continues. The mixture was then heated to 40° C. to dry, followed by addition of the remaining 50% of the croscarmellose sodium, followed by further mixing. Magnesium stearate was then added followed by a final mixing step. The blended powder was then pressed into tablets with the properties listed in Table 25.

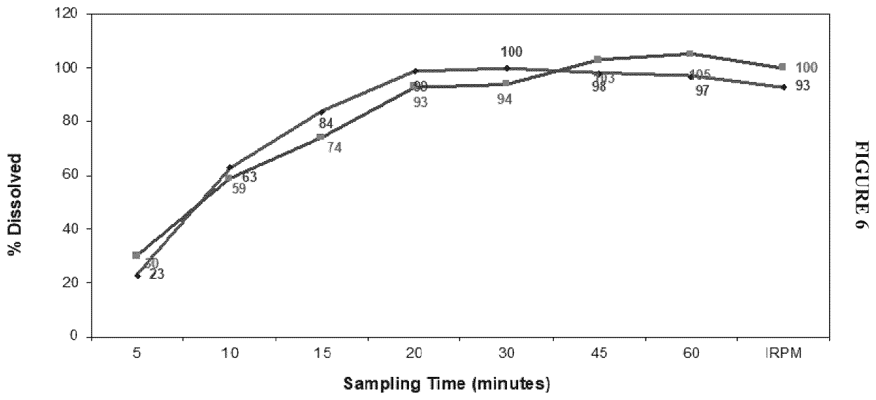

Dissolution of the hydroxychloroquine tablets in water was then assessed and compared to the reference listed drug (RLD) Plaquenil® (i.e., 200 mg hydroxychloroquine sulfate) using a USP No. 2 Apparatus with a paddle at 50 RPM and sampling times of 5, 10, 15, 20, 30, 45 and 60 minutes and infinite time points (FIG. 6). The f2 between the manufactured hydroxychloroquine tablet (i.e., 100 mg hydroxychloroquine sulfate) and the RLD was 58, indicating that the dissolution of the two tablets was comparable and the manufactured tablet met the USP monograph specification of Q≥70% at 60 minutes.

### Cannabidiol (CBD) Sesame Oil Solution

The CBD sesame oil solution comprises 12.5% w/w CBD and 0.02% w/v butylated hydroxytoluene (BHT). The sesame oil containing BHT and sesame oil without BHT were mixed at a ratio of 57:43. The CBD was then dissolved into the oil in two separate and equal additions. The solution was mixed until there are no visible particles, and the density recorded.

The inactive ingredients of the fixed dose combination product are provided in Table 24. Samples of the fixed dose combination product were stored at 5±3° C., 25±2° C./60±5% relative humidity (RH) and 40° C./75% RH and assessed for stability using methods from the US Pharmacopeia (USP) monograph for hydroxychloroquine tablets and high performance liquid chromatography (HPLC) analysis for CBD, hydroxychloroquine and related substances at 0, 1, 2, 3 and 6 months. The 5±3° C., 25±2° C./60±5% RH stored samples are to be assessed every 6 months for 24 months. Dissolution of the CBD and hydroxychloroquine from the fixed dose combination product was also assessed in vitro.

### Fixed Dose Combination Product

The fixed dose combination product is manufactured according to the method described by Salazar Altamaer et al. (U.S. Pat. No. 9,433,584). Briefly, soft gel capsules containing a solid dose form drug encapsulated by a polymer capsule are manufactured in the liquid filled soft gel capsule. For the fixed dose combination product described herein, the hydroxychloroquine tablet is contained within a soft gel capsule of CBD in sesame oil. The resulting soft gel capsules are size 20 oblong soft gel capsules (i.e., 0.986-1.232 cc). Each soft gel capsule contains 100 mg hydroxychloroquine sulfate and 75 mg CBD. The soft gel capsules were manufactured and stored at ambient temperature prior to release analysis. Release of the hydroxychloroquine tablet component was conducted using methods in the USP monograph for hydroxychloroquine sulfate tablets with impurities assessed using HPLC. CBD impurities were assessed using an HPLC method that was developed by SCI Pharmtech based on the one listed in the USP monograph for dronabinol, the synthetic form of THC.

Data from the analysis of the fixed dose combination product are presented in Tables 26 to 28.

### Example 8—Stability of Soft Gel Capsule Fixed Dose Combination Product

The soft gel capsule fixed dose combination product described in Example 7 were packaged into blister packs and a formal stability study with conditions of 5±3° C. and 25±2° C./60±5% RH with time points up to 24 months. Conditions of 40° C./75% RH with time points up to 6 months are currently underway. At least six individual dose units were tested in each assay. Data from the one-month time point of this stability study is presented below in Table 29. Results from all tests met the acceptance criteria at 1-month at all three conditions.

### SUMMARY

Collectively, these data demonstrate that the combination of CBD and hydroxychloroquine synergize to significantly modulate the inflammatory response in vitro and in vivo. The simultaneous administration of these two APIs has been enabled by the encapsulation of CBD in a first capsule and a solid dosage form of hydroxychloroquine in a second capsule, which is incorporated into the first capsule. The present disclosure thus provides a composition that allows for the simultaneous delivery of the synergistic combination of CBD and hydroxychloroquine, regardless of physical-chemical compatibility and/or stabilities liabilities of the two APIs.

TABLE 1
Cannabidiol and related cannabinoids

| | | Chemical |
|---|---|---|
| | | properties/ |
| | | [M + H]⁺ ESI |
| Name | Structure | MS |
| cannabidiol (CBD) | 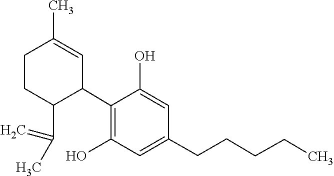 [SMILES: C=C(C)C1CCC(C)=CC1c1c(O)cc(CCCCC)cc1O] | decarboxylation product of CBDA m/z 315.2319 |
| cannabidiolic acid (CBDA) | 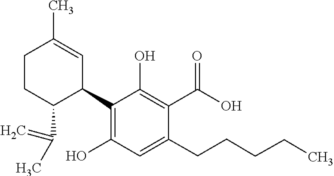 [SMILES: C=C(C)[C@@H]1CCC(C)=C[C@H]1c1c(O)cc(CCCCC)c(C(=O)O)c1O] | m/z 359.2217 |
| cannabigerolic acid (CBGA) | 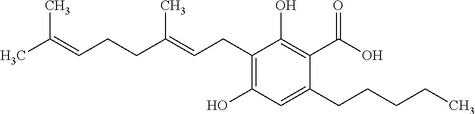 [SMILES: CCCCCc1cc(O)c(C/C=C(\C)CCC=C(C)C)c(O)c1C(=O)O] | m/z 361.2373 |

TABLE 2
Classification of ARDS according to ratio of arterial oxygen
tension and fraction of inspired oxygen (PaO₂:FiO₂)

| Classification | PaO₂:FiO₂ |
|---|---|
| Mild | >200 mmHg and ≤300 mmHg¹ |
| Moderate | >100 mmHg and ≤200 mmHg² |
| Severe | ≤100 mmHg² |

¹As measured on ventilator settings that include positive end-expiratory pressure (PEEP) or continuous positive airway pressure (CPAP) ≥5 cm H₂O;
²as measured on ventilator settings that include PEEP ≥5 cm H₂O.

TABLE 3
Synergistic inhibition of IL-1β, IL-6, TNF-α, IL-1α, and MIP-
1α release by CBD and HCQ in human PBMCs (Donor 1)

| | CBD | | HCQ | | | Ob- | |
|---|---|---|---|---|---|---|---|
| Cytokine | (μg/mL) | FIE | (μg/mL) | FIE | Predicted | served | EOB |
| IL-1β | 5.00 | 0.38 | 0.63 | 0.08 | 0.43 | 0.72 | 0.29 |
| | | | 1.25 | 0.07 | 0.42 | 0.82 | 0.40 |
| | | | 2.50 | 0.08 | 0.43 | 0.88 | 0.45 |
| | | | 5.00 | 0.17 | 0.48 | 0.91 | 0.43 |
| | | | 10.00 | 0.68 | 0.80 | 0.92 | 0.12 |
| | | | 20.00 | 0.81 | 0.88 | 0.98 | 0.10 |
| IL-6 | 5.00 | 0.08 | 0.63 | 0.12 | 0.19 | 0.26 | 0.07 |
| | | | 1.25 | 0.00 | 0.08 | 0.57 | 0.49 |
| | | | 2.50 | 0.10 | 0.17 | 0.43 | 0.26 |
| | | | 5.00 | 0.08 | 0.15 | 0.41 | 0.25 |
| | | | 10.00 | 0.00 | 0.08 | 0.81 | 0.73 |
| | | | 20.00 | 0.12 | 0.19 | 0.93 | 0.74 |
| TNF-α | 5.00 | 0.05 | 0.63 | 0.00 | 0.06 | 0.34 | 0.28 |
| | | | 1.25 | 0.08 | 0.13 | 0.46 | 0.33 |
| | | | 2.50 | 0.05 | 0.11 | 0.57 | 0.47 |
| | | | 5.00 | 0.02 | 0.07 | 0.63 | 0.56 |
| | | | 10.00 | 0.04 | 0.10 | 0.62 | 0.52 |
| | | | 20.00 | 0.30 | 0.34 | 0.90 | 0.57 |
| IL-1α | 5.00 | 0.40 | 0.63 | 0.03 | 0.41 | 0.66 | 0.25 |
| | | | 1.25 | 0.01 | 0.40 | 0.80 | 0.40 |
| | | | 2.50 | 0.12 | 0.47 | 0.85 | 0.38 |
| | | | 5.00 | 0.24 | 0.54 | 0.89 | 0.35 |
| | | | 10.00 | 0.50 | 0.70 | 0.88 | 0.18 |
| | | | 20.00 | 0.54 | 0.72 | 0.96 | 0.24 |
| MIP-1α | 5.00 | 0.12 | 2.50 | 0.00 | 0.12 | 0.26 | 0.14 |
| | | | 20.00 | 0.00 | 0.12 | 0.42 | 0.30 |

FIE—Fractional inhibitory effect;
EOB—Excess over Bliss

TABLE 4
Synergistic inhibition of IL-1β, IL-6, TNF-α, IL-1α, and MIP-
1α release by CBD and HCQ in human PBMCs (Donor 2)

| | CBD | | HCQ | | | Ob- | |
|---|---|---|---|---|---|---|---|
| Cytokine | (μg/mL) | FIE | (μg/mL) | FIE | Predicted | served | EOB |
| IL-1β | 5.00 | 0.13 | 0.63 | 0.06 | 0.19 | 0.34 | 0.15 |
| | | | 2.50 | 0.07 | 0.19 | 0.50 | 0.31 |
| IL-6 | 5.00 | 0.09 | 10.00 | 0.11 | 0.19 | 0.60 | 0.41 |
| | | | 20.00 | 0.47 | 0.52 | 0.83 | 0.31 |
| TNF-α | 5.00 | 0.07 | 5.00 | 0.02 | 0.09 | 0.18 | 0.09 |
| | | | 10.00 | 0.18 | 0.24 | 0.57 | 0.33 |
| IL-1α | 5.00 | 0.28 | 0.63 | 0.07 | 0.33 | 0.51 | 0.18 |
| | | | 1.25 | 0.11 | 0.36 | 0.61 | 0.24 |
| | | | 2.50 | 0.19 | 0.42 | 0.62 | 0.21 |
| | | | 5.00 | 0.49 | 0.63 | 0.74 | 0.11 |
| | | | 10.00 | 0.68 | 0.77 | 0.89 | 0.13 |
| | | | 20.00 | 0.63 | 0.73 | 0.92 | 0.19 |
| MIP-1α | 5.00 | 0.00 | 10.00 | 0.00 | 0.00 | 0.24 | 0.24 |
| | | | 20.00 | 0.00 | 0.00 | 0.62 | 0.62 |

FIE—Fractional inhibitory effect;
EOB—Excess over Bliss

TABLE 5
Synergistic inhibition of inflammatory cytokine release in vivo
following administration of 10 mg/kg CBD and 25 mg/kg HCQ

| | IL-6 | IL-12(p70) | IFN-γ | TNF-α |
|---|---|---|---|---|
| CBD 10 mg/kg | 0.14 | 0.00 | 0.00 | 0.00 |
| HCQ 25 mg/kg | 0.17 | 0.56 | 0.57 | 0.33 |
| Predicted CBD 10 mg/kg HCQ | 0.28 | 0.56 | 0.57 | 0.33 |
| 25 mg/kg | | | | |
| Observed CBD 10 mg/kg HCQ 25 | 0.40 | 0.79 | 0.72 | 0.46 |
| mg/kg | | | | |
| EOB | 0.12 | 0.24 | 0.15 | 0.14 |

EOB—Excess over Bliss

TABLE 6
Synergistic inhibition of inflammatory cytokine release in vivo
following administration of 10 mg/kg CBD and 100 mg/kg HCQ

| | IL-1β | IL-6 | IL-12(p70) | IFN-γ | TNF-α |
|---|---|---|---|---|---|
| CBD 10 mg/kg | 0.34 | 0.14 | 0.00 | 0.00 | 0.00 |
| HCQ 100 mg/kg | 0.01 | 0.22 | 0.72 | 0.74 | 0.55 |
| Predicted CBD 10 mg/kg | 0.35 | 0.33 | 0.72 | 0.74 | 0.55 |
| HCQ 100 mg/kg | | | | | |
| CBD 10 mg/kg HCQ 100 | 0.65 | 0.35 | 0.87 | 0.83 | 0.83 |
| mg/kg | | | | | |
| EOB | 0.30 | 0.03 | 0.15 | 0.09 | 0.28 |

EOB—Excess over Bliss

TABLE 7
Synergistic inhibition of inflammatory cytokine release in vivo
following administration of 25 mg/kg CBD and 25 mg/kg HCQ

| | IL-6 |
|---|---|
| CBD 25 mg/kg | 0.03 |
| HCQ 25 mg/kg | 0.17 |
| Predicted CBD 25 mg/kg HCQ 25 mg/kg | 0.19 |
| CBD 25 mg/kg HCQ 25 mg/kg | 0.33 |
| EOB | 0.15 |

EOB—Excess over Bliss

TABLE 8
Synergistic inhibition of inflammatory cytokine release in vivo
following administration of 25 mg/kg CBD and 100 mg/kg HCQ

| | IL-6 |
|---|---|
| CBD 25 mg/kg | 0.03 |
| HCQ 100 mg/kg | 0.22 |
| Predicted CBD 25 mg/kg HCQ 25 mg/kg | 0.24 |
| CBD 25 mg/kg HCQ 100 mg/kg | 0.36 |
| EOB | 0.13 |

EOB—Excess over Bliss

TABLE 9
Synergistic inhibition of inflammatory cytokine release
in an in vivo model of pulmonary inflammation following
administration of 1 mg/kg CBD and 25 mg/kg

| | IL-1β | IL-6 | MCP-1 | TNF-α |
|---|---|---|---|---|
| CBD 1 mg/kg | 0.03 | 0.15 | 0.07 | 0.23 |
| HCQ 25 mg/kg | 0.20 | 0.08 | 0.09 | 0.14 |
| CBD 1 mg/kg HCQ 25 mg/kg | 0.34 | 0.40 | 0.38 | 0.39 |
| Predicted CBD 1 mg/kg HCQ 25 mg/kg | 0.22 | 0.21 | 0.15 | 0.34 |
| EOB | 0.12 | 0.19 | 0.23 | 0.05 |

EOB—Excess over Bliss

TABLE 10
Synergistic inhibition of inflammatory cytokine release
in an in vivo model of pulmonary inflammation following
administration of 10 mg/kg CBD and 25 mg/kg

| | CXCL-1 |
|---|---|
| CBD 10 mg/kg | 0.14 |
| HCQ 25 mg/kg | 0.35 |
| CBD 10 mg/kg HCQ 25 mg/kg | 0.51 |
| Predicted CBD 10 mg/kg HCQ 25 mg/kg | 0.44 |
| EOB | 0.06 |

EOB—Excess over Bliss

TABLE 11
Relative reduction in white blood cell counts
in an in vivo model of pulmonary inflammation

| | WBC | Neutrophil |
|---|---|---|
| CBD 10 mg/kg | 0.27 | 0.25 |
| HCQ 2.5 mg/kg | 0.11 | 0.10 |
| CBD 10 mg/kg HCQ 2.5 mg/kg | 0.32 | 0.30 |

TABLE 12
Synergistic activity of 1 mg/kg CBD and 2.5
mg/kg HCQ in an in vivo model of colitis

| | | Stool | Macroscopic |
|---|---|---|---|
| | MPO | consistency score | damage score |
| CBD 1 mg/kg | 0.34 | 0.17 | 0.10 |
| HCQ 2.5 mg/kg | 0.00 | 0.00 | 0.05 |
| Predicted CBD + HCQ | 0.34 | 0.17 | 0.15 |
| Observed CBD + HCQ | 0.46 | 0.33 | 0.17 |
| EOB | 0.12 | 0.17 | 0.02 |

EOB—Excess over Bliss

TABLE 13
Relative reduction in Total Colitis Index and
Colon to Body Weight Ratio of 1 mg/kg CBD and
2.5 mg/kg HCQ in an in vivo model of colitis

| | Colon to body weight ratio | Colitis index |
|---|---|---|
| CBD 1 mg/kg | 0.373 | 0.25 |
| HCQ 2.5 mg/kg | 0.287 | 0.27 |
| CBD + HCQ | 0.393 | 0.46 |

TABLE 14
Relative reduction in pulmonary lesion score of 1 mg/kg CBD and
2.5 mg/kg HCQ in an in vivo model of pulmonary inflammation

| | Relative reduction in lesion score |
|---|---|
| CBD 1 mg/kg | 0.18 |
| HCQ 2.5 mg/kg | 0.21 |
| CBD 1 mg/kg + HCQ 2.5 mg/kg | 0.31 |

TABLE 15
Relative reduction in pulmonary lesion score of 10 mg/kg CBD and
25 mg/kg HCQ in an in vivo model of pulmonary inflammation

| | Relative reduction in lesion score |
|---|---|
| CBD 10 mg/kg | 0.29 |
| HCQ 25 mg/kg | 0.37 |
| CBD 10 mg/kg HCQ 25 mg/kg | 0.46 |

TABLE 16
Disease severity score matrix for in vivo model of arthritis

| Score | Condition |
|---|---|
| 0 | Normal |
| 1 | Mild, but definite redness and swelling of the ankle or wrist, |
| | or apparent redness and swelling limited to individual digits, |
| | regardless of the number of affected digits |
| 2 | Moderate redness and swelling of ankle of wrist |
| 3 | Severe redness and swelling of the entire paw including digits |
| 4 | Maximally inflamed limb with involvement of multiple joints |

TABLE 17
Cartilage and bone destruction by pannus formation
scoring matrix in vivo model of arthritis

| Score | Condition |
|---|---|
| 0 | no change |
| 1 | Mild change- pannus formation within cartilage |
| 2 | Moderate change- pannus invasion into cartilage/subchondral bone |
| 3 | Severe change- pannus invasion into the subchondral bone |

TABLE 18
Mononuclear cell infiltration scoring
matrix for in vivo model of arthritis

| Score | Condition |
|---|---|
| 0 | no infiltration |
| 1 | Mild infiltration |
| 2 | Moderate infiltration |
| 3 | Severe infiltration |

TABLE 19
Synergistic activity of 1 mg/kg CBD and 2.5
mg/kg HCQ in an in vivo model of arthritis

| | Clinical | Paw | | Total |
|---|---|---|---|---|
| | score | volume | Pannus | histology |
| | Day 24 | Day 24 | score | score |
| CBD | 0.21 | 0.04 | 0.07 | 0.11 |
| HCQ | 0.18 | 0.04 | 0.07 | 0.15 |
| Pred CBD + | 0.35 | 0.07 | 0.13 | 0.24 |
| HCQ | | | | |
| Obs CBD + | 0.40 | 0.34 | 0.43 | 0.28 |
| HCQ | | | | |
| EOB | 0.05 | 0.26 | 0.30 | 0.03 |

EOB—Excess over Bliss

TABLE 20
Relative reduction in serum cytokine levels of 1 mg/kg
CBD and 2.5 mg/kg HCQ in an in vivo model of arthritis

| | IL-1β | IL-6 |
|---|---|---|
| CBD | 0.31 | 0.50 |
| HCQ | 0.37 | 0.51 |
| CBD + HCQ | 0.25 | 0.36 |

TABLE 21
Comparison of high dose HCQ with low dose HCQ
in combination with 1 mg/kg CBD in reducing disease
severity in an in vivo model of arthritis

| | | CBD 1 mg/kg + |
|---|---|---|
| | HCQ 25 mg/kg | HCQ 2.5 mg/kg |
| Clinical score Day 24 | 0.21 | 0.40 |
| Clinical score Day 30 | 0.26 | 0.31 |
| Paw volume Day 24 | 0.10 | 0.34 |
| Paw volume Day 30 | 0.20 | 0.23 |
| Pannus score | 0.28 | 0.43 |
| Mononuclear cell infiltration | 0.24 | 0.15 |
| Total histology score | 0.26 | 0.28 |
| IL-1B | 0.25 | 0.39 |
| IL-6 | 0.36 | 0.72 |

TABLE 22
Exemplary composition comprising CBD and HCQ

| Active Agent | Amount | Capsule |
|---|---|---|
| CBD solubilized in sesame | 75 mg | Capsule 1 - soft gelatin |
| oil | | capsule |
| Hydroxychloroquine tablet | 20 mg | Capsule 2 - soft gelatin |
| solid dosage form | | capsule |

TABLE 23
Exemplary composition comprising CBD and HCQ

| Active Agent | Amount | Capsule |
|---|---|---|
| CBD solubilized in sesame | 75 mg | Capsule 1 - soft gelatin |
| oil | | capsule |
| Hydroxychloroquine tablet | 100 mg | Capsule 2 - soft gelatin |
| solid dosage form | | capsule |

TABLE 24
List of inactive ingredients in exemplary soft
gel capsule fixed dose combination product

| Component | Inactive ingredient(s) | Function |
|---|---|---|
| Fill (soft gelatine | Sesame oil, NF | Oleaginous vehicle |
| capsule) | Butylated hydroxytoluene | Anti-oxidant |
| | (BHT) | |
| Shell (soft gelatine | Gelatine | Shell material |
| capsule) | Glycerine | Plasticizer |
| | Sorbitol Sorbitan Solution | Plasticizer |
| | Purified Water | Plasticizer/Solvent |
| | Dyes (TBD) | Colour |
| Tablet | Dibasic calcium phosphate | Filler |
| | Magnesium stearate | Lubricant |
| | Pregelatinized Corn Starch | Filler |
| | Croscarmellose sodium | Disintegrant |
| | Opaglos clear coating: | Coating composition |
| | Sodium | |
| | carboxymethylcellulose | |
| | Maltodextrin | |
| | Dextrose monohydrate | |
| | Purified stearic acid | |
| | Sheffcoat White PVA+: | Coating composition |
| | Polyvinyl alcohol | |
| | Titanium dioxide | |
| | Talc | |
| | Soylecithin | |
| | Xanthan rubber | |

TABLE 25
Characteristics of the HCQ tablets in exemplary
soft gel capsule fixed dose combination product

| Parameter | Result |
|---|---|
| % Loss of drying initial | 1.1 |
| % Loss on drying final granule | 1.8 |
| Average Weight (mg) | 170.2 |
| Friability (%) | 0.48 |
| Hardness (kP) | 2.92 |
| Thickness (mm) | 5.58 |
| Diameter (mm) | 6.37 |
| Bulk Density (g/mL) | 0.706 |
| Tapped Density (g/mL) | 0.877 |
| Indice Hausner | 1.24 |
| Carrs Index | 24.18 |

TABLE 26
Analysis of exemplary soft gel capsule fixed dose combination product

| | | | | Acceptance | |
|---|---|---|---|---|---|
| Test | | | Result | Criteria | Conc. |
| Description | | | Meets | Soft gelatin | Meets |
| | | | | capsule of | |
| | | | | green color, | |
| | | | | opaque. It | |
| | | | | contains a | |
| | | | | slightly | |
| | | | | yellow oily | |
| | | | | liquid and a | |
| | | | | white coated | |
| | | | | tablet. Free of | |
| | | | | extraneous | |
| | | | | material. | |
| Adisidine value | | | 0.169 | Report | Meets |
| Peroxide value | | | 3.1 | Report | Meets |
| Weight variation | | | 94.7% | The result | Meets |
| | | | 99.5% | complies if | |
| | | | 97.2% | each of the | |
| | | | 98.6% | individual | |
| | | | 96.0% | weights is | |
| | | | 98.8% | within the | |
| | | | 98.7% | limits 90.0- | |
| | | | 97.2% | 110.0% of | |
| | | | 102.1% | the average | |
| | | | 101.2% | weight. | |
| | | | Average = 98.4% | | |
| HCQ assay | | | 98.0% | 93.0-107.0% | Meets |
| CBD assay | | | 100.3% | 95-110% | Meets |
| | Olivetol | | ND | NMT 0.10% | Meets |
| CBD | CBD-Cet | | ND | NMT 0.10% | Meets |
| individual | Sum of Exo-THC + | | ND | NMT 0.10% | Meets |
| impurities | Δ-8-THC + Δ-9-THC | | | | |
| | Unknown | RRT 0.26 | 0.9 | Report | |
| | impurities | RRT 0.29 | 0.2 | Report | |
| | | RRT 0.32 | 0.01 | Report | |
| | | RRT 0.37 | ND | Report | |
| | | RRT 0.41 | ND | Report | |
| | | RRT 0.46 | ND | Report | |
| | | RRT 1.46 | 0.1 | Report | |

| | | | | |
|---|---|---|---|---|
| | Total CBD impurities | 1.1 | NMT 2.0% | Meets |
| HCQ | Desthyl hydroxychloroquine | <0.1% | Report | |
| individual | Hydroxychloroquine acetate | <0.1% | Report | |
| impurities | Sulfohydroxychloroquine | <0.1% | Report | |
| | Chloroquine related compound | <0.1% | Report | |
| | A | | | |
| | Any individual unspecified | <0.1% | NMT 0.2% | Meets |
| | impurity | | | |
| | Total hydroxychloroquine | <0.1% | NMT 2.0% | Meets |
| | impurities | | | |

ND = none detected; NMT = not more than.

TABLE 27
Dissolution of HCQ from exemplary soft gel capsule
fixed dose combination product using the USP
monograph UV-spectrophotometric method

| | Capsule | 343 nm | | 348 nm | | 345 nm | |
|---|---|---|---|---|---|---|---|
| Condition | No. | 60 | IRPM | 60 | IRPM | 60 | IRPM |
| With | 1 | 95 | 96 | 96 | 98 | 95 | 96 |
| immersion | 2 | 98 | 98 | 100 | 101 | 100 | 101 |
| device | 3 | 98 | 99 | 98 | 100 | 96 | 99 |
| | 4 | 100 | 98 | 99 | 99 | 97 | 99 |
| | 5 | 111 | 110 | 107 | 108 | 106 | 107 |
| | 6 | 97 | 101 | 100 | 101 | 98 | 100 |
| | Average | 100 | 100 | 100 | 101 | 99 | 100 |
| | SD | 5.8 | 4.8 | 3.6 | 3.5 | 4.0 | 3.5 |
| With | 1 | 100 | 104 | 99 | 97 | 99 | 95 |
| sinker | 2 | 101 | 106 | 101 | 102 | 100 | 101 |
| | 3 | 99 | 111 | 98 | 99 | 95 | 98 |
| | 4 | 101 | 101 | 98 | 99 | 97 | 98 |
| | 5 | 97 | 101 | 98 | 100 | 97 | 99 |
| | 6 | 101 | 100 | 99 | 101 | 97 | 99 |
| | Average | 100 | 104 | 99 | 100 | 98 | 98 |
| | SD | 1.5 | 4.0 | 1.2 | 1.6 | 1.8 | 2.1 |

IRPM = Infinity RPM mode; SD = standard deviation.

TABLE 28
Dissolution of CBD from exemplary soft gel capsule fixed dose
combination product using the USP No. 2 (paddle apparatus)

| Capsule No. | Q (%) | IRPM (%) |
|---|---|---|
| 1 | 97.0 | 97.7 |
| 2 | 94.9 | 94.9 |
| 3 | 99.6 | 100.2 |
| 4 | 95.9 | 96.8 |
| 5 | 99.1 | 99.7 |
| 6 | 98.3 | 97.5 |
| Average | 98 | 98 |
| SD | 1.7 | 1.8 |

IRPM = Infinity RPM mode; Q = the amount of dissolved active ingredient (i.e., CBD); SD = standard deviation.

TABLE 29
Stability of exemplary soft gel capsule fixed dose combination after one month storage

| | | | Accept. | | | 25 ± 2° C.; | 40° C. |
|---|---|---|---|---|---|---|---|
| Test | | | Crit. | Release | 5 ± 3° C. | 60 ± 5% RH | 75% RH |
| HCQ Assay (% listed) | | | 93.0- | 98.0 | 97.2 | 98.2 | 97.2 |
| | | | 107.0% | | | | |
| Adisidine value | | | Report | 0.2 | 10 | 9 | 11 |
| Peroxide value | | | Report | 3.1 | 4 | 8 | 8 |
| HCQ dissolution at 60 min (Q %) | | | Q ≥ 70% | 100 | 99 | 87 | 85 |
| CBD assay (% listed) | | | 95- | 100.3 | 100.1 | 100.3 | 100.2 |
| | | | 110% | | | | |
| CBD dissolution at 60 min | | | Q ≥ 70% | 97 | 89 | 95 | 97 |
| CBD | Olivetol | | NMT | ND | ND | ND | ND |
| individual | | | 0.10% | | | | |
| impurities | CBD-Cet | | NMT | ND | ND | ND | ND |
| | | | 0.10% | | | | |
| | Sum of Exo-THC + Δ-8- | | NMT | ND | ND | ND | ND |
| | THC + Δ-9-THC | | 0.10% | | | | |
| | Unknown | RRT | Report | 0.9 | 0.8 | 0.8 | 0.9 |
| | impurities | 0.26 | | | | | |
| | | RRT | Report | 0.2 | 0.2 | 0.2 | 0.2 |
| | | 0.29 | | | | | |
| | | RRT | Report | 0.01 | 0.02 | 0.01 | 0.02 |
| | | 0.32 | | | | | |
| | | RRT | Report | ND | ND | ND | 0.1 |
| | | 0.37 | | | | | |
| | | RRT | Report | ND | ND | ND | 0.2 |
| | | 0.41 | | | | | |
| | | RRT | Report | ND | ND | ND | 0.03 |
| | | 0.46 | | | | | |
| | | RRT | Report | 0.1 | ND | ND | 0.1 |
| | | 1.46 | | | | | |

| | Total CBD impurities | NMT | 1.1 | 1.1 | 1.1 | 1.6 |
|---|---|---|---|---|---|---|
| | | 2.0% | | | | |
| HCQ | Desthyl | Report | <0.1% | <0.1% | <0.1% | <0.1% |
| individual | hydroxychloroquine | | | | | |
| impurities | Hydroxychloroquine | Report | <0.1% | <0.1% | <0.1% | <0.1% |
| | acetate | | | | | |
| | Sulfohydroxychloroquine | Report | <0.1% | <0.1% | <0.1% | <0.1% |
| | Chloroquine related | Report | <0.1% | <0.1% | <0.1% | <0.1% |
| | compound A | | | | | |
| | Any individual | NMT | <0.1% | <0.1% | <0.1% | <0.1% |
| | unspecified impurity | 0.2% | | | | |
| | Total | NMT | <0.1% | <0.1% | <0.1% | <0.1% |
| | hydroxychloroquine | 2.0% | | | | |
| | impurities | | | | | |

ND = none detected;
NMT = not more than;
Q = the amount of dissolved active ingredient (i.e., CBD, HCQ)

1. A composition comprising:
a. cannabidiol (CBD) or a pharmaceutically acceptable salt or derivative thereof; and
b. a solid dosage form of hydroxychloroquine or a pharmaceutically acceptable salt thereof,
wherein the CBD or a pharmaceutically acceptable salt or derivative thereof is encapsulated by a first capsule and the solid dosage form of hydroxychloroquine or a pharmaceutically acceptable salt thereof is encapsulated by a second capsule, wherein the second capsule is at least partially encapsulated by the first capsule.

2. The composition of claim 1, wherein the CBD or a pharmaceutically acceptable salt or derivative thereof is solubilized in a liquid solvent selected from the group consisting of an oil, an alcohol, propylene glycol and glycerol.

3. The composition of claim 2, wherein the liquid solvent is an oil selected from the group consisting of hemp seed oil, olive oil, caprylic/capric triglyceride (MCT) oil, sunflower oil and sesame seed oil.

4. The composition of claim 1, wherein the first capsule is a soft gelatin capsule.

5. The composition of claim 2, wherein the second capsule is a capsule that is not solubilized by the liquid solvent.

6. The composition of claim 5, wherein the second capsule is selected from the group consisting of a polymer capsule, a soft gelatin capsule and a cellulose capsule.

7. The composition of claim 6, wherein the second capsule is a polymer capsule.

8. The composition of claim 1, wherein solid dosage form is selected from the group consisting of a tablet, a powder, a microgranule, a nanoparticle and a pellet.

9. The composition of claim 8, wherein the solid dosage form is a tablet.

10. The composition of claim 1, wherein the second capsule is incorporated within the first capsule.

11. The composition of claim 1, wherein the CBD or a pharmaceutically acceptable salt or derivative thereof is a synthetic compound.

12. The composition of claim 1, which comprises at least about 25 mg CBD or a pharmaceutically acceptable salt or derivative thereof.

13. The composition of claim 12, which comprises from about 25 mg to about 100 mg CBD or a pharmaceutically acceptable salt or derivative thereof.

14. The composition of claim 13, which comprises about 75 mg CBD or a pharmaceutically acceptable salt or derivative thereof.

15. The composition of claim 1, which comprises at least about 10 mg hydroxychloroquine or a pharmaceutically acceptable salt thereof.

16. The composition of claim 15, which comprises from about 10 mg to about 200 mg hydroxychloroquine or a pharmaceutically acceptable salt thereof.

17. The composition of claim 16, which comprises about 20 mg hydroxychloroquine or a pharmaceutically acceptable salt thereof.

18. The composition of claim 16, which comprises about 100 mg hydroxychloroquine or a pharmaceutically acceptable salt thereof.

19-20. (canceled)

21. A method for the treatment of an inflammatory condition comprising administering the composition of claim 1 to a subject in need thereof.
